(19) United States
(12) Patent Application Publication
(10) Pub. No.: US 2022/0246275 A1
(43) Pub. Date: Aug. 4, 2022

(54) SYSTEMS AND METHODS FOR GENERATING A CONGENITAL NOURISHMENT PROGRAM

(71) Applicant: KPN INNOVATIONS, LLC., LAKEWOOD, CO (US)

(72) Inventor: Kenneth Neumann, LAKEWOOD, CO (US)

(73) Assignee: KPN INNOVATIONS, LLC., LAKEWOOD, CO (US)

(21) Appl. No.: 17/164,558

(22) Filed: Feb. 1, 2021

(51) Int. Cl. G16H 20/60; G06N 3/08; G16H 50/30

(52) U.S. Cl. CPC G16H 20/60; G16H 50/30; G06N 3/08

(57) ABSTRACT

A system for generating a congenital nourishment program includes a computing device configured to acquire at least a congenital factor relating to a subject, retrieve a congenital parameter related to the congenital factor, determine, using the congenital parameter, a nourishment identifier, wherein generating the nourishment identifier includes identifying, using the congenital parameter, a phenotype associated with the at least a congenital factor, generating, using the phenotype, a congenital relationship, wherein the congenital relationship relates at least an effect of at least a nourishment identifier on the phenotype, and determining the nourishment identifier as a function of the at least an effect, identify, using the nourishment identifier, at least a nutrition element, and generate a consumption model using the at least a nutrition element.

### FIELD OF THE INVENTION

The present invention generally relates to the field of nutrition programming for addressing congenital disorders. In particular, the present invention is directed to systems and methods for generating a congenital nourishment program.

### BACKGROUND

Major congenital malformations occur in at least 2% of human births. The origins of most malformations remain difficult to elucidate, and possibly involve a combination of genetic and environmental factors, or genotype-environment interaction (GxE). Although malformations frequently occur in isolation, they also occur in combination. Isolated congenital defects are currently known to be due to mutations in many genes, some associated with physiological signal transduction amenable to nutritional intervention.

### SUMMARY OF THE DISCLOSURE

In an aspect, a system for generating a congenital nourishment program for addressing congenital disorders including a computing device configured to acquire at least a congenital factor relating to a subject, determine, using the at least a congenital factor, a nourishment identifier, wherein generating the nourishment identifier includes identifying, using the at least a congenital factor, a phenotype, generating, using the phenotype, a congenital relationship, wherein the congenital relationship relates at least an effect of at least a nourishment identifier on the phenotype, and determining the nourishment identifier as a function of the congenital relationship, identify, using the nourishment identifier, at least a nutrition element, and generate a consumption model using the at least a nutrition element.

In another aspect, a method for generating a congenital nourishment program for addressing congenital disorders including acquiring, by the computing device, at least a congenital factor relating to a subject, determining, by the computing device, using the at least a congenital factor, a nourishment identifier, wherein generating the nourishment identifier includes identifying, using the at least a congenital factor, a phenotype, generating, using the phenotype, a congenital relationship, wherein the congenital relationship relates at least an effect of at least a nourishment identifier on the phenotype, and determining the nourishment identifier as a function of the congenital relationship, identifying, by the computing device, using the nourishment identifier, at least a nutrition element, and generating, by the computing device, a consumption model using the at least a nutrition element.

These and other aspects and features of non-limiting embodiments of the present invention will become apparent to those skilled in the art upon benefit of the disclosure in its entirety of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating a system for generating a congenital nourishment program;

FIG. 2 is a block diagram illustrating a machine-learning module;

FIG. 3 is a block diagram of a nourishment program database;

FIGS. 4A-B are diagrammatic representations of an exemplary embodiment of a congenital profile;

FIG. 5 is a diagrammatic representation of a congenital nourishment program;

FIG. 6 is a diagrammatic representation of a user device;

FIG. 7 is a block diagram of a method for generating a congenital nourishment program; and

FIG. 8 is a block diagram of a computing system that can be used to implement any one or more of the methodologies disclosed herein and any one or more portions thereof.

The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for generating a congenital nourishment program for congenital disorders. In an embodiment, system includes a computing device configured to receive congenital factors relating to a subject. Congenital factors may include data from physiological sensors, experimental testing results, genotypic and phenotypic analysis, biomarkers, and the like. Computing device is configured to retrieve a congenital parameter corresponding to the subject. Congenital parameter may include a plurality of parameters organized into a genetic and environmental fingerprint of the subject. Computing device may generate congenital parameters by using a machine-learning algorithm indicating possible mathematical relationships linking values to congenital factors, where values may be compared to threshold values indicating healthy ranges. Computing device may classify an individual based on a congenital parameter to a congenital disorder, such as a diagnosis based on a combination of genetic and environmental factors. Computing device is configured to generate a nutraceutical model which associated congenital parameter to a phenotype, correlating the phenotype to effects of nutrition on the phenotype. Computing device is configured to determine nutrition elements as a function of the nutraceutical model. Computing device may generate a congenital nourishment program using a linear programming function to generate an ordering of nutrition elements as a function of a consumption model. In an embodiment, computing device may provide nutrition elements to a subject device via a graphical user interface and provide a nourishment metric that relates the congenital state of the subject.

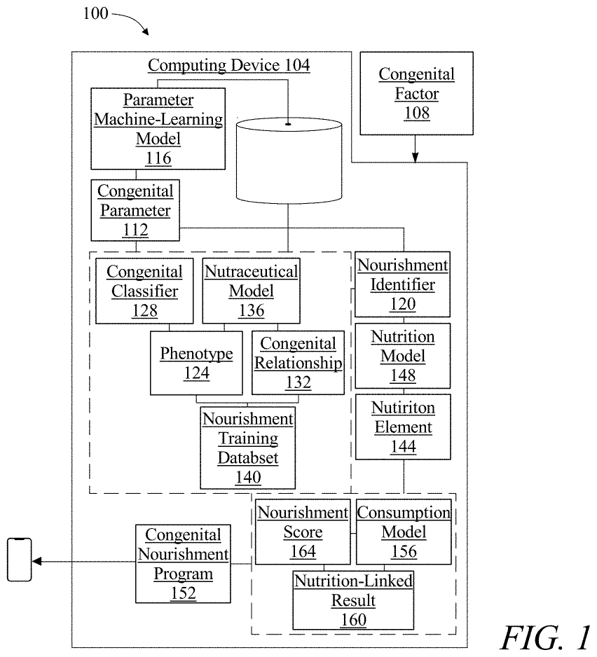

Referring now to FIG. 1, an exemplary embodiment of a system 100 for generating a congenital nourishment program for addressing congenital disorders using machine-learning is illustrated. System includes a computing device 104. Computing device 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device 104 may include a single computing device operating independently, or may include two or more computing device operating in concert, in parallel, sequentially or the like; two or more computing devices may be included together in a single computing device or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus, or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software, and the like) may be communicated to and/or from a computer and/or a computing device. Computing device 104 may include but is not limited to, for example, a computing device or cluster of computing devices in a first location and a second computing device or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device, which may operate in parallel, in series, redundantly, or in any other manner used for distribution of tasks or memory between computing devices. Computing device 104 may be implemented using a “shared nothing” architecture in which data is cached at the worker, in an embodiment, this may enable scalability of system 100 and/or computing device.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Continuing in reference to FIG. 1, computing device is configured to acquire at least a congenital factor from a subject. A “congenital factor,” as used in this disclosure, is an environmental, biological, and/or chemical substance or process that is indicative of or contributing to congenital disorder. Congenital factor 108 may include biological molecules and/or biomarkers existing within a normal cell, a stressed cell, disease state cell, and/or a specific response of the body indicative of disorder, malfunction, and/or dysfunction. Congenital factor 108 may be present at birth. Acquiring congenital factor 108 may include receiving a result of one or more tests relating to the subject, such as a blood panel, genetic testing, X-ray, proteomics analysis, and the like. Congenital factor 108 may include test results of screening and/or early detection of disease, diagnostic procedures, prognostic indicators from other diagnoses, such as Sickle cell anemia, from predictors identified in a medical history, and physiological data and data relating to biomolecules associated with and/or found within the subject such as physiological parameters including CXCL9, CD4+, CD25+, IFN-γ+, CD4+, IL5+, DPD, Protein C, albumin, IgG, Kidney injury molecule-1, N-acetyl-B-D-glucosaminidase, B2 microglobulin, heart-type fatty acid binding protein, macrophage migration inhibitory factor, neutrophil gelatinase-associated lipocalin, monocyte chemotactic protein-1. A person skilled in the art, having the benefit of the entirety of this disclosure, will be aware of various additional tests and/or data that may be used and or received as congenital factor 108.

Continuing in reference to FIG. 1, congenital factor 108 may include results and or analysis enumerating the identification of nucleic acids. Congenital factor 108 may include the presentation of single nucleotide polymorphisms (SNPs), mutations, chromosomal deletions, inversions, translocation events, and the like, in genetic sequences, for instance as isolated from the body, that may be indicative of congenital disease. Congenital factor 108 may include epigenetic factors such as patterns of microRNAs (miRNAs), aberrations in gene expression, and the like. Congenital factor 108 may include hematological analysis including results from T-cell activation assays, abnormal nucleation of white blood cells, white blood cell counts, concentrations, recruitment and localization, and the like. Congenital factor 108 may be received as a function of a subject indicating a prior diagnosis, treatment received, among other data indicated in a medical history, physician's assessment, and the like. Congenital factor 108 may include any symptoms, side effects, and co-morbidities associated with and relating to treatment regimens, recovery from injury and/or illness, and the like. Congenital factor 108 may be received and/or identified from a biological extraction of a subject, which may include analysis of a physical sample of a subject such as blood, saliva, hair, stool, and the like, without limitation and as described in U.S. Nonprovisional application Ser. No. 16/886,647, filed May 28, 2020, and entitled, “METHODS AND SYSTEMS FOR DETERMINING A PLURALITY OF BIOLOGICAL OUTCOMES USING A PLURALITY OF DIMENSIONS OF BIOLOGICAL EXTRACTION SUBJECT DATA AND ARTIFICIAL INTELLIGENCE,” the entirety of which is incorporated herein by reference.

Continuing in reference to FIG. 1, congenital factor 108 may include environmental factors that do not originate from subject. Environmental factors may include risk factors associated with the parent of the subject, for instance and without limitation, insufficient folic acid, drinking during pregnancy, smoking, exposure to certain medications (such as thalidomide), advanced maternal age (AMA), and the like. Congenital factor 108 may include acute and/or chronic exposure to subject and/or parent of subject to carcinogens, teratogens, chemical irritants, pharmaceuticals, heavy metals, pesticides, radiation exposure, foreign particulates such as silica dust, asbestos, allergens, among other environmental factors. Congenital factor 108 may include data regarding intrauterine infection and maternal infection during and/or before pregnancy such as by Zika Virus, syphilis, rubella, among others. Congenital factor 108 may include biologics that may traverse the placental barrier, including immunoglobulins from the mother. For instance and without limitation, enzyme-linked immunosorbent assay (ELISA), PCR, and/or antigen testing of a parent may indicate recent exposure to a pathogen that may result in congenital abnormality, wherein such data may be congenital factor 108 which informs the phenotype of subject, despite the data origination form outside the subject.

Continuing in reference to FIG. 1, congenital factor 108 may be organized into training data sets. “Training data,” as used herein, is data containing correlations that a machine learning process, algorithm, and/or method may use to model relationships between two or more categories of data elements. For instance, and without limitation, training data may include a plurality of data entries, each entry representing a set of data elements that were recorded, received, and/or generated together; data elements may be correlated by shared existence in a given data entry, by proximity in a given data entry, or the like. Multiple data entries in training data may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data according to various correlations; correlations may indicate causative and/or predictive links between categories of data elements, which may be modeled as relationships such as mathematical relationships by machine learning processes as described in further detail below.

Continuing in reference to FIG. 1, congenital factor 108 may be used to generate training data for a machine-learning process. A “machine learning process,” as used in this disclosure, is a process that automatedly uses a body of data known as “training data” and/or a “training set” to generate an algorithm (such as a collection of one or more functions, equations, and the like) that will be performed by a machine-learning module to produce outputs given data provided as inputs; this is in contrast to a non-machine learning software programing where the commands to be executed are determined in advance by a subject and written in a programming language, as described in further detail below.

Continuing in reference to FIG. 1, congenital factor 108 may be stored and/or retrieved by computing device 104, without limitation, as a relational database, a key-value retrieval database such as a NOSQL database, or any other format or structure for use as a database that a person skilled in the art would recognize as suitable upon review of the entirety of this disclosure. Congenital factor 108 training data may alternatively or additionally be implemented using a distributed data storage protocol and/or data structure, such as a distributed hash table and the like. Congenital factor 108 training data may include a plurality of data entries and/or records, as described above. Data entries may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries of congenital factor 108 data may be stored, retrieved, organized, and/or reflect data and/or records as used herein, as well as categories and/or populations of data consistent with this disclosure.

Continuing in reference to FIG. 1, computing device is configured to retrieve a congenital parameter related to the subject. A “congenital parameter,” as used in this disclosure, is a datum that enumerates congenital factor data in the subject. Congenital parameter 112 may include thresholds, ranges of numerical values, binary determinations, and the like, which summarize the current state of congenital disease, risk management of secondary infection, disease, and the like, of the subject. Congenital parameter 112 may include a numerical value that is compared to a threshold value for congenital factor 108 in healthy cohorts. In such an instance, a numerical value may be assigned to the threshold value and the congenital parameter 112 includes a determination about the deviation of the factor from the healthy range. Congenital parameter 112 may include information relating to the presence of mutations, polymorphisms, protein truncations, and the like, wherein a score may be provided according to the functionality of an enzyme, signaling pathway, among other physiological phenomena.

Continuing in reference to FIG. 1, for instance and without limitation, a congenital parameter 112 may enumerate concentrations of blood metabolites relative to a threshold value in a cohort of healthy adults. In such an instance, the congenital parameter may be represented by a small positive numerical value for biomarkers within the “healthy” or “normal” range, large positive numerical value for concentration indicating exceptional health, and negative values indicating a deficiency or alarming increase relative to the normal threshold range or value. Congenital parameter 112 may be biomarker-specific, for instance and without limitation, a numerical value for each of 100+ types of physiological categories, where each numerical value communicates a likelihood that a congenital factor 108 relates to a normal/healthy state, an abnormal state, a particular disorder, among other categorizations. Congenital parameter 112 may include any medical, physiological, biological, chemical, and/or physical determination about the current state of congenital disease, including projected, future likelihood for disease or complication resulting from a congenital disorder and/or dysfunction. Congenital parameter 112 may include qualitative and/or quantitative metrics of the presence of symptomology, development of disease, biomarkers indicative of disease, biomarkers classified to subcategories, and the like. Congenital parameter 112 may include qualitative determinations, such as binary “yes”/“no” determinations for particular types, “normal”/“abnormal” determinations about the presence of and/or concentration of congenital factor 108, for instance as compared to a normalized threshold value of a biomarker among healthy adults. Congenital parameter 112 may include a plurality of congenital parameters, wherein congenital parameters are quantitative determinations such as a “congenital scoring”, which may include any metric, parameter, or numerical value that communicates a value relating to a biomarker. Congenital parameter 112 may include congenital parameters that are mathematical expressions relating the current congenital state. Congenital parameter 112 may include mathematical expression interrelating combinations of biomarkers as they may relate to clinical significance. Computing device 104 may retrieve congenital parameter 112 from a database, as described in further detail below.

Continuing in reference to FIG. 1, retrieving congenital parameter 112 may include a process of searching for, locating, and returning congenital parameter 112 data. For example, congenital parameter 112 may be retrieved as documentation on a computer to be viewed or modified such as files in a directory, database, and the like. In non-limiting illustrative embodiments, computing device 104 may locate and download congenital parameter 112 via a web browser and the Internet, receive as input via a software application and a subject device, via a telemedicine platform from a physician, lab technician, shared data storage point, and the like.

Continuing in reference to FIG. 1, retrieving congenital parameter 112 may include receiving data via a graphical user interface. A “graphical user interface,” as used in this disclosure, is any form of a subject interface that allows a subject to interface with an electronic device through graphical icons, audio indicators, text-based interface, typed command labels, text navigation, and the like, wherein the interface is configured to provide information to the subject and accept input from the subject. Graphical user interface may accept input, wherein input may include an interaction with a subject device. A subject device, as described in further detail below, may include computing device 104, a “smartphone,” cellular mobile phone, desktop computer, laptop, terminal, tablet computer, internet-of-things (TOT) device, wearable device, among other devices. Subject device may include any device that is capable for communicating with computing device 104, database, or able to receive data, retrieve data, store data, and/or transmit data, for instance via a data network technology such as 3G, 4G/LTE, 5G, Wi-Fi (IEEE 802.11 family standards), and the like. Subject device may include devices that communicate using other mobile communication technologies, or any combination thereof, for short-range wireless communication (for instance, using Bluetooth and/or Bluetooth LE standards, AirDrop, Wi-Fi, NFC, and the like), and the like.

Still referring to FIG. 1, retrieving the congenital parameter 112 related to the subject may include training a parameter machine-learning model with training data that includes a plurality of data entries correlating congenital factors to a plurality of congenital parameters and generating the congenital parameter as a function of the parameter machine-learning model and the at least a congenital factor. Computing device 104 may generate congenital parameter 112 as a function of the parameter machine-learning model and at least a congenital factor 108. Parameter machine-learning model 116 may include any machine-learning process, algorithm, and/or model as performed by machine-learning module, described in further detail below. Relationships observed in training data to enumerate congenital factors for congenital parameter 112 may be used to statistically relate congenital disorder to other maladies for which no directly observable data exists. For instance and without limitation, a combination of biomarkers relating to interleukins, prostaglandins, cytokines, complement pathway molecules such as C-reactive proteins, tumor necrosis factor, cellular stress markers, among other biomarkers, may indicate the presence of metabolic difficulties and nutritional deficiencies, of which there is no direct biochemical observation of.

Continuing in reference to FIG. 1, training data for parameter machine-learning model 116 may include congenital factors 108 organized into training data sets, as described above, including results from biological extraction samples, health state questionnaires regarding symptomology, medical histories, physician assessments, lab work, and the like, which may be entered by users and/or received electronically from other devices. Training data may be retrieved from a database, as described in further detail below. Congenital parameter 112 training data may originate from the subject, for instance via a questionnaire and a subject interface with computing device 104, for subject to provide medical history data and/or symptoms. Receiving congenital parameter training data may include receiving whole genome sequencing, gene expression patterns, and the like, for instance as provided by a genomic sequencing entity, hospital, database, the Internet, and the like. Congenital parameter 112 training data may include raw data values recorded and transmitted to computing device 104 via a wearable device such as a bioimpedance device, ECG/EKG/EEG monitor, physiological sensors, blood pressure monitor, blood sugar and volatile organic compound (VOC) monitor, and the like. Congenital parameter 112 training data may originate from an individual other than subject, including for instance a physician, lab technician, nurse, dietician, strength coach, psychologist, and the like. It is important to note that training data for machine-learning processes, algorithms, and/or models used within system 100 herein may likewise originate from any source described for congenital parameter 112 training data.

Continuing in reference to FIG. 1, parameter machine-learning model 116 may include any machine-learning algorithm such as K-nearest neighbors algorithm, a lazy naïve Bayes algorithm, among other algorithms, machine-learning process such as supervised machine-learning, unsupervised machine-learning, or method such as neural nets, deep learning, and the like. Parameter machine-learning model 116 may be trained to derive an equation, function, series of equations, or any mathematical operation, relationship, or heuristic, that can automatedly accept an input, such as congenital factor(s) 108, and correlate, classify, or otherwise calculate an output, such as congenital parameter(s). Parameter machine-learning model 116 may include individual functions, derived for unique relationships observed from the training data for each congenital factor 108 and/or combination thereof. In non-limiting illustrative examples, the parameters involved in a variety of physiological tests, as identified above, may be retrieved from a database, such as a repository of peer-reviewed research (e.g. National Center for Biotechnology Information as part of the United States National Library of Medicine), and the congenital parameter machine-learning model 116 may derive an algorithm which determines an average and statistical evaluation (mean±S.D.) calculated from the data, across which the subject's parameters may be compared. In such an example, parameter machine-learning model 116 may derive an algorithm according to the data used to derive the average and statistical evaluation changes as a function of the subset of data to which the subject is to be compared, for instance and without limitation, based on age, weight, sex, genetic profiling, nutrition deficiency, symptomology, prior diagnoses, and the like.

Continuing in reference to FIG. 1, computing device 104 is configured to generate, using the congenital parameter 112, a nourishment identifier. A “nourishment identifier,” as used in this disclosure, is a label identifying a biologically active compound whose consumption is intended for addressing congenital disorder. A nourishment identifier 120 may include a nutrient identify and may be related to a congenital relationship, as described in further detail below. Nourishment identifier 120 may relate to supplementary use of oral digestive enzymes. In such an instance, nourishment identifier 120 may relate to units of enzyme activity, specific activities, and the like. Nourishment identifier 120 may include probiotics which may also have merit as anti-congenital disorder measures. In such an instance, nourishment identifier 120 may include the identities of bacterial isolates, colony forming units (CFU/mL), and the like. Nourishment identifier 120 may include mass amounts of micronutrients such as vitamins, minerals, sugars, amino acids, trace elements, hormones, electrolytes, such as N-acetyl cysteine, selenium, folic acid, vitamin D, bicarbonate, copper, and the like. Nourishment identifier 120 may include values relating to phytonutrients and plant-based biomolecules such as chlorophyll, antioxidants such as the carotenoids (α-carotene, β-carotene, lycopene, lutein, cryptoxanthin), and the like. Nourishment identifier 120 may contain biologically active compounds that are not typically considered as part of recommended daily nutrients, nor are they intended to provide appreciable amounts of calories, such as phytonutrients, nutraceuticals, antioxidants, and the like; for instance and without limitation, allium and bioactive ingredients present in cruciferous vegetables such as broccoli sprouts, which are known sources of antioxidants such as sulforaphane, polyphenols present in fruits, vegetables, seeds, nuts, legumes, and the like.

Continuing in reference to FIG. 1, generating the nourishment identifier includes identifying, using the congenital parameter 112, a phenotype associated with the at least a congenital factor. A “phenotype,” as used in this disclosure, is a composite observable characteristic or trait of subject. Phenotype 124 may include a congenital disorder, anomaly, and the like, such as hearing defects, trisomy 18 (Edward's syndrome), trisomy 21 (down syndrome), trisomy 13 (Patau syndrome), cleft palate, spina bifida, phenylketonuria, glutamate carboxypeptidase II mutation, pyloric stenosis, congenital hip dislocation, anencephaly, hypoplasia, Meckel's diverticulum, and the like. Phenotype 124 may include a genotype-environment interaction (GxE). Phenotype 124 may include any diagnosis (current disorder) and/or prognosis (predicted difficulty, future diagnosis, outcome, and the like) associated with congenital factor 108. Phenotype 124 may include identifiers associated with disorders, conditions, symptoms, and the like, which may correspond with categorization. Phenotype 124 may include a predictive classification, where a subject may be considered reasonably healthy at birth, does not harbor congenital factor(s) 108 indicative of obvious current congenital disorder but may include data that indicates a phenotype 124 with which they may be most closely categorized to, and/or an imminent categorization. Congenital parameter 112 may have associated with it an identifier, such as a diagnostic label, that corresponds to a phenotype 124. Phenotype 124 may be stored and/or retrieved from a database.

Continuing in reference to FIG. 1, identifying the phenotype may include training a congenital classifier using training data which includes a plurality of data entries of congenital factors from a subset of categorized subjects. A “congenital classifier,” as used in this disclosure, is a machine-learning classifier that sorts congenital parameter 112 to phenotype 124. A classifier may include a machine-learning model, such as a mathematical model, neural net, or program generated by a machine learning algorithm known as a “classification algorithm,” as described in further detail below. Congenital classifier 128 may be generated by a classification machine-learning process, which may include any machine-learning algorithm, process, and/or model described herein performed by a machine-learning module, as described in further detail below. Classification machine-learning process may generate congenital classifier 128 using training data. Training data may include identification of particular biomarkers, threshold values associated with each, and/or congenital parameters that correlate to a particular disorder category. Training data may include a plurality of data values relating biomarkers to disorders, where each biomarker is assigned a variable, and the variables may be used in an empirical formula for determining the presence of a disorder or applying a particular diagnosis. Training data may include environmental factors correlated to congenital disorders, including magnitude of associated effect. For instance and without limitation, maternal environment interaction may be used to determine relationships between intrauterine relationships to environmental factors which may classify a subject to a congenital disorder category. Congenital classifier 128 may sort inputs, such as congenital parameter 112, into categories or bins of data, such as classifying the data into phenotype 124, outputting the bins of data and/or labels associated therewith.

Continuing in reference to FIG. 1, training data for congenital classifier 128 may include a set of congenital factor 108 data as it relates to classes of disorder types, disease types, anticipated nutritional deficiency, metabolic disorder, and the like. For instance and without limitation, training data may include ranges of congenital factor 108 as they correlate to various degrees of anemia, wound healing, immunological function, and the like. Such training data may include congenital factor 108 as it relates to phenotype 124 for subsets of a plurality of subjects, segmented according to subject characteristics such as by environmental factor, pathogen exposure, smoking, exercise, diet, age, sex, alcohol consumption, ethnicity, nutritional deficiency, co-morbidities, and the like. Training data may be used by classification machine-learning process to train a classifier to derive relationships present in the data that may result in a machine-learning model that automatedly classifies a subject to a phenotype 124 as a function of the data present in their congenital parameter(s) 112. Training data may originate from any source described herein; for instance training data may be retrieved from a database, retrieved via a network such as the Internet, peer-reviewed research repository, clinical data, subject input data, wearable device, physiological sensor, medical history data, and the like.

Continuing in reference to FIG. 1, congenital classifier 128 may be configured to output at least a datum that labels or otherwise identifies a set of data that are clustered together, found to be close, relate to one another via a metric, scoring, probability, and the like, as described in further detail below. Machine-learning module, as described in further detail below, may generate a classifier using a classification algorithm, defined as a process whereby computing device and/or any module and/or component operating thereon derives a classifier from training data. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, a congenital parameter 112 training data classifier may classify elements of training data to elements that characterizes a sub-population, including subset of congenital factor 108 such as gene expression patterns, SNPs, mutations, and epigenetic markers as it relates to a variety of disorder types and/or other analyzed items and/or phenomena for which a subset of training data may be selected.

Continuing in reference to FIG. 1, classifying congenital parameter 112 to phenotype 124 may include generating a threshold value of a congenital parameter 112, wherein the threshold value indicates a value for comparing the congenital parameter 112 for determining the presence of a congenital anomaly. Computing device 104 may search, identify, and retrieve a threshold value, for instance via a web browser and the Internet. In non-limiting illustrative examples, threshold value may indicate a numerical value, or range of numerical values, such as 11-12.5 seconds for prothrombin time (PT) and/or patrial thromboplastin time (PTT) which indicates a normal amount of time for blood clotting. In such an example, deviations in clotting time may indicate increased risk of blood clot (quicker times) or increased risk of hemophilia or lack of blood clotting ability (longer times), which may be emblematic of congenital bleeding disorders such as hemophilia, which may have been diagnosed at birth or gone undiagnosed. Generating a threshold value may include calculating a threshold value. For instance, computing device 104 may retrieve PT and PTT data points for subsets of infants, toddlers, adolescents, young adults, and the like, exhibiting no nutritional deficiency, exhibiting healthy weight to determine a threshold range of values for such biomarkers that may represent more accurate comparison for classification. In such an example, a time of 13.0 seconds may be “normal” among geriatrics, but instead relates to a time one standard deviation above average for a younger cohort, potentially indicating an issue. Computing device 104 may then classify subjects into cohorts of alike subsets according to comparison along the threshold. Classification in this manner may result in defining subsets of subjects for more accurately determining the presence of congenital disorder among the population, and more accurately identifying the presence of nutrition-related congenital functions.

Continuing in reference to FIG. 1, identifying the phenotype 124 may include classifying the congenital factor 108 to the phenotype 124 using the congenital classifier 128 and identifying the phenotype 124 as a function of the classifying. Classification using congenital classifier 128 may include identifying which set of categories (phenotype 124) an observation (congenital factor 108) belongs. Alternatively or additionally, the observation may include a congenital parameter 112. Classification may include clustering based on pattern recognition, wherein the presence of congenital factor 108, such as genetic indicators, symptoms, and the like, identified by congenital parameter 112 relate to a particular phenotype 124. Such classification methods may include binary classification, where the congenital parameter 112 is simply matched to each existing phenotype 124 and sorted into a category based on a “yes”/“no” match. Classification done in such a manner may include weighting, scoring, or otherwise assigning a numerical value to elements in congenital parameter 112 as it relates to each disorder type and assign the subject to a phenotype 124 that results in the ‘highest’ score, depending on the criteria for highest. Such a score may represent a likelihood, probability, or other statistical evaluation that relates to the classification into phenotype 124.

Continuing in reference to FIG. 1, classifying may include classifying the congenital parameter 112 (and/or congenital factor 108) to a nutrition-linked congenital disorder category. A “nutrition-linked congenital disorder category,” as used in this disclosure, is a congenital disorder categorization that indicates a category which is sensitive to nutritional modification. Nutrition-linked congenital disorder category may include identifying a category of current disorders that are not averse to nutritional modification in that they may be addressed, at least in part, by varying nutrition levels in the subject. Correspondingly, classification in this manner may lead to identification of congenital disorders which may remain unaffected by nutrition. A nutrition-linked congenital disorder category may include for instance and without limitation disorders with vitamin synthesis pathways, such as with folate synthesis and NAD synthesis, where supplementation of B vitamin complexes and B vitamin intermediates may “cure” the deficiency where the disorder categorization may be changed with sufficient, sustained nutrient supplementation, and/or symptoms may be effectively managed. Such classification may include identifying congenital factor 108 which are resistant to nutritional changes and identifying which can be addressed with nutritional modification, altering dietary habits, nutrient supplementation, and the like.

Continuing in reference to FIG. 1, classifying may include classifying the congenital parameter 112 to a nutrition-linked disorder prevention category. A “nutrition-linked disorder prevention category,” as used in this disclosure, is a congenital disorder categorization for which nutrients may act as a preventative measure. Nutrition-linked disorder prevention category may include a category which will occur, or is imminent, according to the congenital parameter 112 of the subject which may be prevented or ameliorated from nutritional modification. A nutrition-linked disorder prevention category may include a risk for developing blood cancer in the future, where the risk of developing the blood cancer may be reduced, or delayed, from nutritional intervention. Classification to such a category may include identifying congenital factor(s) 108 which may be modified, over time, with sustained, chronic nutrient manipulation.

Continuing in reference to FIG. 1, determining the nourishment identifier 120 includes generating, using the phenotype, a congenital relationship, wherein the congenital relationship relates at least an effect of at least a nourishment identifier on the phenotype. A “congenital relationship,” as used in this disclosure, is a range of nutrient amounts that are predicted to have an effect on phenotype 124; congenital relationship may include a range of nutrient amounts that are predicted to have an effect on a congenital factor 108 that results in a concomitant effect on phenotype 124. Congenital relationship 132 may include numerical values, ranges, functions, and/or any other mathematical arrangement describing nutrient amounts and their effect on congenital disorder.

Continuing in reference to FIG. 1, determining the at least a congenital relationship 132 may include generating a nutraceutical model using a machine-learning process and training data which includes a plurality of data entries correlating effects of nourishment identifiers 132 to phenotypes 124. A “nutraceutical model,” as used in this disclosure, is a machine-learning model trained with training data to derive mathematical relationships between nutrients and congenital disorders. Nutraceutical model 136 may include any machine-learning algorithm and/or process performed by a machine-learning module, as described in further detail below. Nutraceutical model 136 may be trained with training data retrieved from a database, via a web browser to the Internet, or from any source described herein. Training data may include a plurality of nutrients and nutrient combinations correlated to effects on various congenital malformations, disorders, and/or anomalies. Such training data may include numerical values indicating levels of effectiveness for each disorder, ranges of nutrient amounts as a function of symptom severity, age, and the like. Training data may include a plurality of data entries including ranges of values of nutrients, for a variety of nutrient types and classes such as water-soluble vitamins, fat-soluble vitamins, transition metal nutrients, trace metals, alkali earth metal nutrients, phytonutrients, essential and non-essential amino acids, and the like, which are related to an effect on congenital disorders. Training data may originate from a database, for instance, containing a plurality of nutrition facts of a plurality of food items, menu items, and the like, where the identities of the items are linked to data elements describing the items' nutritional content. Training data may originate from computing device 104, for instance using a web browser and the internet to retrieve nutritional data. Training data may originate as input, for instance as subject input via a graphical user interface or input from an electronic device such as a user device, IoT device, and the like. Such training data may relate to the magnitude of effect of acute and chronic nutrient deficiencies over time. Training data may be segmented by cohort, where congenital relationship 132 for large sets (>1,000+) of healthy subjects is compared to congenital relationships of cohorts of subjects classified by phenotype 124 to calculate congenital relationship 132 mismatch. Categorization of training data may be performed by a classifier, as described herein. The subject may be classified to a training data set cohort using a classifier, as described herein. Nutraceutical model 136 may derive any number of congenital relationships between nourishment identifiers 132 and congenital factors 108, congenital parameters 112, and/or phenotype 124. Mathematical relationships may be equations, formulas, functions, and the like, derived from relationships observed in the training data which may be used to automatedly accept inputs of phenotype 124 and generate outputs of nourishment identifiers 120, wherein the magnitude of the nutrient amount and its associated effect may be included in the relationship and be numerically described in the output.

Continuing in reference to FIG. 1, generating the nutraceutical model 136 may include any machine-learning model type, algorithm, and/or process using training data. Training data may include observations and/or relationships relating genetic variations to responses to bioactive ingredients, such as pharmaceutical products. For instance and without limitation, pharmaceutical products metabolized by N-acetyltransferase such as the muscle relaxant suxamethonium chloride may result in a variety of efficacies of such drugs and similarly metabolized products among the population. Approximately one in 3500 Caucasian individuals has a less efficient variant of the enzyme (butyrylcholinesterase) that metabolizes suxamethonium chloride. Consequently, the pharmaceutical product's effect is prolonged, with slower recovery from surgical paralysis. An enzyme system known as the cytochrome P450 oxidases provides the body with an inborn system for clearing xenobiotics (chemicals not normally produced by or expected to be present in the body). The cytochrome P450 oxidases are involved in pharmaceutical product metabolism, and genetic variations in their pathways may affect large populations of individuals, especially in how metabolism effects the build-up of oxidative stresses, nutritional deficiencies, among others. The thiopurines and thiopurine methyl transferase enzyme system has been involved in one test for a genetic variation in drug metabolism that had a clinically important consequence. This system metabolizes 6-mercaptopurine and azathioprine, two thiopurine drugs used in a range of clinical indications, from leukemia to autoimmune diseases. In humans with thiopurine methyl transferase deficiency, thiopurine metabolism proceeds by other pathways, one of which leads to production of an active thiopurine metabolite that is toxic to the bone marrow. The frequency of this mutation is one in 300 people. These individuals need about 6-10% of the standard dose of the drug. If treated inadvertently with the full dose of the pharmaceutical product, these individuals are at risk for severe bone marrow suppression. For such humans, genotype predicts clinical outcome, which may be considered a prerequisite for an effective pharmacogenetic test. Such pharmacogenetic approach may be applied to nutrigenomics for congenital disorders in humans in generating the nutraceutical model.

Continuing in reference to FIG. 1, congenital relationship 132 may be described by a vector, for instance with a direction and magnitude that describes if the disorder will improve or worsen, and by which amount according to the nutrient amount and the current congenital relationship of the subject. Congenital relationships may include a function including a series of values that may be stored in a table in a database for all nutrient amounts to provide intended effect on a variety of congenital disorder categories 120. In this way, for each subject classified to a phenotype 124, a congenital relationship 132 may be retrieved from the database. Congenital relationship 132 may include empirical formulas, for instance retrieved by computing device 104 using a web browser and the Internet, database, and the like, which inform correlations between nutrient consumption and congenital disorder. Persons skilled in the art, upon receiving the benefit of this disclosure in its entirety, may appreciate that nutraceutical model 136 may determine increasingly complicated relationships between 100+ different nutrient types and the many phenotypes 124 subjects may be classified.

Continuing in reference to FIG. 1, computing device may be further configured to generate a nourishment training dataset from a plurality of congenital relationship 132 outputs of the nutraceutical model 136. A “nourishment training dataset,” as used in this disclosure, is a machine-learning training data set comprising outputs from nutraceutical model 136. Nourishment training dataset 140 may include a plurality of phenotypes 124 output by congenital classifier 128 correlated to congenital relationships wherein the relationships include nourishment identifiers and nutrient effects. In this way, computing device may iteratively improve upon the generation of nourishment identifiers throughout the life of the subject by generating training data sets of machine-learning outputs described herein. For instance and without limitation, this may be used to detect when nutrients have lost their effectiveness and/or when a dietary change is necessary.

Continuing in reference to FIG. 1, nourishment training dataset 140 may be used with nutraceutical model 136 to derive an equation, system of equations, function, and/or any mathematical relationship which may inform a nutrient that may address congenital factor(s) 108. Nutrient amounts may be calculated using the mathematical relationship derived from the nourishment training dataset 140 which may assign variables to age, body weight, severity of symptom, among other factors, and provide per-user, customized nutrient amounts. Combinatorial effects may arise where supplementation of a first nutrient is somehow affected by a second nutrient, leading to the identification of nutrient combinations. Such nutrient combinations may provide synergistic effects where absorption of a first nutrient is boosted by a second. For instance, adsorption of calcium can be boosted by vitamin C and plant-based iron, vitamin D and calcium, foods high in fat with fat soluble vitamins, etc. Novel nutrient-nutrient relationships in response to genotype-phenotype relationships may be identified in this way.

Continuing in reference to FIG. 1, computing device 104 may generate the nourishment identifier 120 as a function of the at least an effect. Identifying congenital relationship 132 may include determining a respective effect of each nutrient of a plurality of congenital relationships on the phenotype 124. An “effect of a nutrient,” as used in this disclosure, is a change, consequence, and/or result in at least a congenital factor 108, congenital parameter 112, and/or phenotype 124 due to consumption of an amount of a nutrient. A “nutrient amount,” as used in this disclosure, is a numerical value(s) relating to a nourishment identifier 120. Nutrient amount may include mass amounts of a vitamin, mineral, macronutrient (carbohydrate, protein, fat), a numerical value of calories, amounts of phytonutrients, antioxidants, probiotics, nutraceuticals, bioactive ingredients, and the like. An effect of a nutrient amount may include “no effect”, “negligible effect”, and/or “no calculated effect”. Determining an effect of a nutrient may include determining how a congenital factor 108 may change, such as an increase/decrease in concentration of a metabolic intermediate, blood analyte, gene expression, and the like, according to a particular amount of nutrient. For instance and without limitation, such a determination may include calculating the effect of chronic, sustained nutrient amounts in a diet for weeks and/or months on epigenetic factors, blood serum levels of biomarkers, and the like.

Continuing in reference to FIG. 1, determining a respective effect of each nutrient amount of the plurality of nutrients may include retrieving the effects of the nutrient amount on the phenotype 124. Computing device 104 may search for a nutrient effect using each congenital factor 108, and/or combination thereof contributing to the disorder, to locate and retrieve effects correlated to nutrients targeting a congenital factor 108. Retrieving an effect of a nutrient may include retrieving a hypothesis about the outcome for a subject after consuming a nutrient amount and/or amount of a combination of nutrients. Such a hypothesis may include an equation, function, among other mathematical forms, for instance derived from empirical relationships between a nutrient and the physiological integrity of an organ, biological system, and the like. Retrieving an effect may include retrieving from a database, a research repository, or the like. Retrieving an effect may include, for instance, searching using the congenital parameter 112, a web browser, and the Internet, for a plurality of effects that nutrients may have. Retrieving an effect may include searching using the phenotype 124 for an effect of a nutrient. In some embodiments, retrieving an effect may include calculating at least an effect, for instance by deriving a function from training data using a machine-learning algorithm.

Continuing in reference to FIG. 1, in non-limiting illustrative examples, determining an effect of a nutrient may include calculating if a change in phenotype 124 may arise from adding and/or removing a nutrient from a subject's diet. For instance and without limitation, changing a phenotype 124 from “congenital dyserythropoietic anemia (CDA)” to “normal serum iron level” with decreasing dietary heme-based iron from reduction and/or elimination of certain animal and plant-based nutrition elements 140, such as beef, pork, chicken, veal, fish, lentils, chickpeas, tofu, and the like. CDA is a rare congenital blood disorder marked by ineffective erythropoiesis and resulting from a decrease in the number of red blood cells (RBCs) in the body and a less than normal quantity of hemoglobin in the blood. This may result in excessive serum iron level accumulation where the subject may undergo chelation therapy to reduce iron concentration from toxic levels. In such an instance, iron consumption in the diet is related to iron accumulation in the blood; therefore, a relationship may be derived in subjects exhibiting congenital factors related to CDA which would modulate nutrients to reduce iron accumulation, where a precise balance is struck wherein a minimal iron level is maintained, but toxic accumulation is avoided. Although chelation therapy may not be eliminated by this strategy, the frequency of the chelation therapy may be markedly reduced.

Continuing in reference to FIG. 1, calculating an effect of a nutrient may include a mathematical operation, such as subtraction, addition, and the like. Calculating an effect of a nutrient may include retrieving an empirical equation that describes relationships between a nutrient and congenital factor 108, test results, congenital parameter, and the like. Calculating an effect of a nutrient may include deriving an algorithm, function, or the like, for instance using a machine-learning process and/or model. Calculating such an effect using machine-learning may include training data that includes a plurality of nutrients as it relates to effects on congenital factor 108 and/or phenotype 124.

Continuing in reference to FIG. 1, determining a respective effect of each nutrient amount of the plurality of nutrients may include generating a machine-learning model. Training data may include nutrient amounts correlated to their effect on the human body. For instance and without limitation, supplementation of amounts of fat-soluble vitamins, water-soluble vitamins, trace elements, minerals, electrolytes, among other nutrient categories in the diet may be correlated to renal function, liver function, vision integrity, bone mineral density, and the like. Such training data may originate from a database, research repository, clinical data, physician, plurality of subjects, or any other source described herein. Computing device 104 may generate a machine-learning model with such training data to derive an equation and/or function which describes relationships observed in the training data. Computing device 104 may then automatedly derive a respective effect for each nutrient, or nutrient combination, wherein the effect may become increasingly defined by parameters relating to the type of congenital disorder in the subject. The effect may also be related to an equation wherein, the magnitude of effect may be determined for all amounts of the nutrient. In this way, a particular nutrient amount may be determined based on the magnitude of effect desired.

Continuing in reference to FIG. 1, calculating a plurality of nutrient amounts may include generating training data using a plurality of predicted effects of the plurality of nutrient amounts. Training data may include retrieving effects on the functioning of biological systems according to nutrient effects. Computing device 104 may generate training data which includes nutrient amounts correlated to phenotype 124 the nutrient is intended to target by retrieving effects and linking data elements to one another and storing in a database. Training data may include nutrient identities correlated to particular disorders, for instance vitamin B12, copper, and vitamin C deficiency may correlate to cytopenia and hematologic symptoms. Training data may include nutrient combinations from peer-reviewed studies correlated to congenital symptoms, for instance combination supplementation of folate, vitamin B12, and methylmalonic acid (MMA) may address congenital symptoms specifically in subjects adhering to strict ovo-vegetarian diets. Training data may include identified nutrient deficiencies in cohorts of subjects with particular lifestyle indicators, diet types, pre-existing conditions, co-morbidities, and the like. Training data may include nutrient surpluses in cohorts of subjects with no congenital disorder to compare against in identifying novel nutrients and combinations which may prevent symptomology arising from a congenital anomaly. Training data may originate from any source described herein, for instance and without limitation, from a physician, via subject input from a plurality of subjects, a database, as described in further detail below, research repository, wearable device, physiological sensor, and the like.

Continuing in reference to FIG. 1, calculating the plurality of nutrient amounts may include generating a machine-learning model according to the training data, wherein training data includes a plurality of data entries that correlates the magnitude of nutrient effect to a plurality of nutrient amounts for each phenotype 124. Such a machine-learning model may include any machine-learning process, algorithm, and/or model as performed by machine-learning module described in further detail below. The machine-learning model may be trained with training data that includes a plurality of data entries that includes nutrient effects, including the magnitude of effect, and effects of nutrient combination correlated to phenotype 124. Data may be correlated to phenotype 124 in that it is correlated to particular congenital factor 108, symptom alleviation, may be found in a subset of healthy adults, among other correlations. In this way, such a machine-learning model may derive equations, functions, among other heuristics, which describe relationships observed in the training data regarding the full spectrum of nutrient amounts targeted to the subject's phenotype 124 and/or congenital factor 108.

Continuing in reference to FIG. 1, computing device 104 may calculate nutrient amounts, for instance, by retrieving a default amount from a database. Computing device 104 may retrieve standard nutrient amounts, such as from a standard 2,000 calorie diet, and alter the amount according to a numerical scale associated with congenital factor 108 in the congenital parameter 112. Such a calculation may include a mathematical expressing using operations such as subtraction, addition, multiplication, and the like, for instance an equation that assigns a variable to the subject's body weight, congenital parameters summarized in congenital parameter 112, and retrieve a default value of a vitamin and alter the amount using the mathematical expression. Alternatively or additionally, such a calculation may involve deriving a loss function, vector analysis, linear algebra, system of questions, among other mathematical heuristics, depending on the granularity of the process. Deriving such a process for calculating nutrient amounts may include machine-learning, as described herein. Nutrient amounts may include threshold values, or ranges of values, for instance and without limitation, between 80-120 mg vitamin C per 24 hours, wherein the range changes as a function of congenital parameter 112. Nutrient amounts may be calculated as heat maps (or similar mathematical arrangements), for instance using banding, where each datum of congenital parameter 112 elicits a particular congenital relationship 132 of a particular nutrient amount or set of amounts. In non-limiting illustrative examples, such a calculation may include querying for and retrieving a standard amount of water-soluble vitamins for a healthy adult, for instance as described below in

Table 1:

TABLE 1

| Nutrient | Amount |
|---|---|
| Vitamin C | 60 mg/day |
| Thiamin (B1) | 0.5 mg/1,000 kcal; 1.0 mg/day |
| Riboflavin (B2) | 0.6 mg/1,000 kcal; 1.2 mg/day |
| Niacin (B3) | 6.6 NE/1,000 kcal; 13 ND/day |
| Vitamin B6 | 0.02 mg/1 g protein; 2.2 mg/day |
| Vitamin B12 | 3 μg/day |
| Folic Acid | 400 μg/day |

Continuing in reference to FIG. 1, in reference to Table 1 above, wherein NE is niacin equivalent (1 mg niacin, or 60 mg tryptophan), mg (milligram), kcal (1000 kcal=1 Calorie), and μg (microgram). Computing device 104 may store and/or retrieve the above standard nutrient amounts, for instance via a database. The amounts may be re-calculated and converted according to a subject's congenital parameter 112. For instance, these amounts may relate to an average BMI, healthy adult male, for any range of calories, but may be adjusted according to unique subject-specific congenital factor 108. In non-limiting illustrative examples, a geriatric woman who adheres to a 1,400 Calorie/day diet, with complete blood count (CBC) test results indicating decreased WBC, RBC, and hemoglobin concentration, may be provided custom target nutrient values that deviate from the default values. In such an example, nutrient amounts may be curated according to the identified risk factors (congenital factor 108) and the above nutrient amounts may be recalculated, where some amounts may increase, some may decrease, and some may remain constant according to a congenital relationship 132.

Continuing in reference to FIG. 1, calculating nutrient amounts may include deriving a weighting factor to adjust, or otherwise re-calculate, an amount. Weighting factor may be determined by computing device 104, for instance, by querying for vitamin amounts according to data inputs identified in the congenital parameter 112. For instance in non-limiting illustrative examples, if congenital parameter 112 indicates decreased energy, pancytopenia, failure to thrive, and hypotonia. Management of coagulopathy may include treatments such as fresh frozen plasma, pRBC, platelet transfusions, and vitamin K supplementation. In such an instance, further diagnostic testing may reveal markedly low serum B12 with elevated MMA and homocysteine levels, mildly low folate and copper levels, and markedly low vitamin D level. Pancytopenia and coagulopathy may improve in as soon as within 7 days on oral vitamin K, 2 doses of vitamin B12, vitamin ADEK, and copper. In such a symptomology manifestation, issues may be found to be due to milk protein allergy that existed from birth. Therefore, standard predetermined diets (potentially including breast feeding) which may increase protein or suggest animal products may have worsened the condition due to elevated MMA, homocysteine, and milk protein allergy. The consumption schedule for the individual may be altered such that the subject is weaned off oral vitamin K, vitamin ADEK, copper, and vitamin B12 supplementation once congenital factor 108 indicate normal CBC. In such an instance, relationships may be identified in nutrient amounts relating to addressing the phenotype 124 of such an individual, specifically in supplementing the diet with specific foods items rich in those vitamins and minerals, whereas it may be inversely associated with consumption of certain animal products such as red meat and/or dairy products. Additionally, vitamins found in such foods from organic sources may be superior from nonorganic sources, such as from commercially available supplements, from a bioavailability standpoint. Per-subject pharmacokinetics, rates of metabolism and/or adsorption of nutrients may differ subject-to-subject, which may negate the effectiveness of proscribing particular predetermined diet types and nutrition elements 140 to subjects. In such an instance, computing device 104 may account for such details using machine-learning to derive more specific nutrient amount calculations and to more accurately calculate the amounts by which to increase/decrease nutrients found in such foods as evidence by the presence of congenital factor 108. Therefore, computing device 104 may derive weighting factors to account for particular genotype, phenotype, epigenetic factors, organic vs non-organic sources, and the nutrition element types with which the nutrients may originate.

Continuing in reference to FIG. 1, in non-limiting illustrative examples, computing device 104 may use a machine-learning process to perform a machine-learning algorithm to derive per-subject pharmacokinetics, for instance of vitamin B6. The machine-learning algorithm may accept an input of numerical values including the total amount of protein consumed (in grams), total amount of vitamin B6 consumed (in mg) per day in a diet, and serum levels of the vitamin B6 vitamer, pyridoxal-5-phosphate, over the course of a month, and derive the rates of metabolism, or how ‘well’ the subject is obtaining the vitamin from nutrition elements 140 and adsorbing vitamin B6. In other words, the algorithm may derive a function such as using linear regression, vector quantization, least squares, among other algorithms, that describes the pharmacokinetics for that particular subject regarding what amount of vitamin B6 consumed, per amount of dietary protein, results in what corresponding amount of bioactive vitamin compound, as measured by the blood vitamer from a biological extraction. Such a function, derived from machine-learning, may then be used by computing device 104 with an input of the congenital parameter 112, which enumerates congenital factor 108, to calculate an output which is a more accurate, customized, per-subject nutrient amount of vitamin B6. Persons skilled in the art, upon benefit of this disclosure in its entirety, may appreciate that this process may be repeated for the full spectrum of nutrients, both required as part of a diet and not required as part of a diet, to control for specific metabolic differences in a population. Alternatively or additionally, such a process may identify new gut malabsorption issues related to nutrients that are recommended but not typically associated with disease.

Continuing in reference to FIG. 1, additionally, in non-limiting illustrative examples, computing device 104 may relate the concentrations of the metabolic products related to vitamins (e.g. vitamers), minerals, phytonutrients, probiotics, antioxidative compounds, biologically active ingredients, prodrugs, and the like, to their effective concentrations in tissues. For instance, computing device 104 may additionally search and retrieve data that relates the blood levels of the vitamin B6 vitamer, pyridoxal-5-phosphate, to the effective concentrations of vitamin B6 in the liver, which is particularly sensitive to aberrations in hematologic function. Computing device 104 may store and/or retrieve values in a “look-up table”, or graph a relationship as a mathematical function, among other ways of representing a data structure that relates the data identified in the search. Alternatively or additionally, computing device 104 may derive a function, for instance using machine-learning, which correlates the concentration of a compound in a particular biological extraction, such as blood, to varying amounts in tissues such as breast tissue, liver, kidneys, and the like This may prove helpful in calculating nutrient amounts as a function of subject consumption to specific target nutrient amount quantities within a particular organ/tissue according to the input data in the congenital parameter 112.

Continuing in reference to FIG. 1, computing device 104 may determine nutrient amounts by receiving input from the subject and calculating nutrition inputs. “Nutritional input,” as used in this disclosure, is an amount of a nutrient consumed by a subject. Nutritional input may be received and/or calculated, for instance and without limitation, as described in Ser. No. 16/911,994, filed Jun. 25, 2020, titled “METHODS AND SYSTEMS FOR ADDITIVE MANUFACTURING OF NUTRITIONAL SUPPLEMENT SERVINGS,” the entirety of which is incorporated herein by reference. Computing device 104 may receive nutritional input from subject. Nutritional input, for instance and without limitation, may include food items that have associated nutrition facts, wherein computing device 104 may calculate, weight, or otherwise modify, the nutritional input from the subject, such as with a weighting factor. This results in accurate, per-subject nutritional input. Such nutritional input may be used to determine target congenital relationship 132. For instance and without limitation, if subject regularly consumes <2,000 calorie diets due to elevated visceral fat content, hip-to-waist-ration, and increased body mass index (BMI), nutraceutical model 136 may determine congenital relationships 128 that may address phenotype 124 and is feasible within the daily target calorie level.

Continuing in reference to FIG. 1, computing device 104 is configured to determine, using the nourishment identifier 120, at least a nutrition element. A “nutrition element,” as used in this disclosure, is an item that includes a nutrient amount intended to be consumed by subject. Nutrition element 144 may include alimentary elements, such as meals (e.g. chicken parmesan with Greek salad and iced tea), food items (e.g. French fries), grocery items (e.g. broccoli), health supplements (e.g. whey protein and multivitamin), beverages (e.g. orange juice), and the like. Nutrition element 144 may be “personalized” in that nutrition elements are curated in a guided manner according to congenital factor 108, congenital parameter 112, phenotype 124, subject-designated symptoms, food allergies and/or intolerances, subject preferences, and the like. Nutrition element 144 may include a specific dietary category, such as a “ketogenic diet”, “low glycemic index diet”, “Paleo diet”, among others. Nutrition element 144 may include custom meals, recipes, and/or beverage which may not be traditionally found, such as a “health shake” which includes a unique, proprietary blend of ingredients that is optimized for a particular subject.

Continuing in reference to FIG. 1, determining the at least a nutrition element 140 may include generating a nutrition model 148 using training data including a plurality of data entries of nourishment identifiers correlating to nutrition elements and determining the at least a nutrition element 140 as a function of the nutrition model and the nourishment identifier 120. Nutrition model 148 may be generated using a nutrition machine-learning process including any machine-learning algorithm, process, and/or model as performed by a machine-learning module, as described in further detail below. Nutrition model 148 may be trained using training data which may originate from any source, as described herein, for instance and without limitation retrieved from a database, retrieved via a web browser and the Internet, peer-reviewed research repository, clinical data, subject input data, wearable device, physiological sensor, medical history data, and the like. Training data may include a plurality of data entries including ranges of values of nutrients, for a variety of nutrient types and classes such as water-soluble vitamins, fat-soluble vitamins, transition metal nutrients, trace metals, alkali earth metal nutrients, phytonutrients, essential amino acids, and the like, which are related to nutrition elements that contain varying amounts of each. Such training data may be used to determine which to mix-and-match into combinations to arrive at which nutrition elements should be consumed within particular ranges of time (within a meal, a day, week, and the like) to arrive at target congenital relationship 132. Training data may be segmented by cohort, where congenital relationship 132 for large sets (>1,000+) of healthy subjects is compared to congenital relationships of cohorts of subjects classified by phenotype 124 to calculate nutrition elements 140 which may close the gap between the cohorts.

Continuing in reference to FIG. 1, computing device 104 may determine the at least a nutrition element 144 as a function of the nutrition machine-learning process and the at least a congenital relationship 132. Nutrition model 148 may be trained to derive mathematical relationships observed in the training data to automatedly accept congenital relationship 132 inputs and calculate an output that is at least a nutrition element 144, of a plurality of nutrition elements 140. Nutrition element 144 outputs may include sets, or combinations, of nutrition elements that are categorized as packets necessary to reach nutrient targets. For instance and without limitation, nutrition elements may include at least one element from a “fruit” category, “vegetable” category, and “grain category”, that may be arranged such that a meal will achieve at least a first congenital relationship 132, without over-contributing to a second congenital relationship 132. Nutrition model 148 may determine an amount of nutrition element 144 where the amount is related to how much should be consumed in a meal, day, week, and the like. Persons skilled in the art, upon receiving the benefit of this disclosure in its entirety, may appreciate that nutrition model 148 may identify thousands or more nutrition elements for each phenotype 124 that should be avoided or included into a diet according to any number of congenital relationships 128.

Continuing in reference to FIG. 1, determining at least a nutrition element 144 may include retrieving a plurality of nutrition elements 140 from a data repository as a function of at least a congenital relationship 132. Data repository may include any data structure such as a congenital nourishment program database, as described in further detail below. Identifying a plurality of nutrition elements 140 may include retrieving nutrition elements that correspond to a congenital relationship 132 of the plurality of nutrient amounts, according to data present in congenital parameter 112. For instance and without limitation, congenital parameters that indicate nutrition-linked congenital disorder category may result in retrieval of nutrition elements 140 that contain a minimal nutrient content according to congenital relationship 132. Computing device 104 may accept an input of at least a congenital relationship 132 and retrieve nutrition elements 140 by searching a database for nutrition elements according to the nutrient and its range of values. Computing device 104 may accept an input of congenital relationship 132 and may search using a web browser and the Internet for nutrition elements 140 according to the nutrient and its amount.

Continuing in reference to FIG. 1, identifying the plurality of nutrition elements 140 may include identifying the nutrition elements 140 according to the phenotype 124. Computing device 104 may accept an input of a congenital disorder category and retrieve a nutrition element 144, or category of nutrition elements according to the classification. Identifying nutrition element 144 according to phenotype 124 may include querying, for instance using a web browser and the Internet, for foods, supplements, bioactive ingredients, and the like, which are correlated with a particular phenotype 124. For instance and without limitation, computing device 104 may organize a search for foods intended for “low platelet concentration”, wherein an entire diet may be crafted around target congenital relationships 128 and the categorization of the congenital parameter 112 to a phenotype 124 that relates to the need for increasing platelets. In such an example, the nutrition elements 140 are outputs generated from an input search criteria of “low platelet concentration” and its associated congenital relationships 128. The output elements become “personalized” as they are arranged into daily, weekly, monthly, and the like, individual meals and/or consumption schedule according to a subject's particular calculated nutrient amounts. The phenotype 124 may serve as a filtering step, wherein a search is guided by the congenital parameter 112 as it was classified to a disorder type.

Continuing in reference to FIG. 1, determining the plurality of nutrition elements 140 may include generating combinations of located nutrition elements as a function of fulfilling a plurality of nutrient amounts. In this way, custom meals may be generated according to the nutritional needs calculated of a congenital disorder. Computing device 104 may identify the plurality of nutrition elements 140 by using congenital relationship 132 as an input and generating combinations, lists, or other aggregates of nutrition elements 140 necessary to achieve congenital relationship 132. For instance, computing device 104 may use a template congenital relationship 132 of ‘200 mg vitamin C’ and build a catalogue of nutrition elements 140 until the 200 mg vitamin C value is obtained. Computing device 104 may perform this task by querying for food items, for instance from a menu, grocery list, or the like, retrieving the vitamin C content, and subtracting the value from the congenital relationship 132. In non-limiting illustrative examples, computing device 104 may identify orange juice (90 mg vitamin C/serving; 200 mg-90 mg=110 mg) for breakfast, Brussel sprouts (50 mg vitamin C/serving; 110 mg-50 mg=60 mg) for lunch, and baked potato (20 mg vitamin C/serving) and spicy lentil curry (40 mg vitamin C/serving; 60 mg−(20 mg+40 mg)=0 mg) for dinner. In such an example, computing device 104 may search according to a set of instructions such as subject preferences, allergies, dietary restrictions, and the like, provided by a physician, medical history, subject input, among other sources, and subtract each identified nutrition element 144 nutrient from congenital relationship 132 until a combination of nutrition elements 140 that represents a solution is identified. Once a solution is found, computing device 104 may generate a file of nutrition elements 140 and store in a database, as described in further detail below. In this way, computing device 104 may generate customized meals, health shakes, recipes, and the like, which may be retrieved from a database as identified for a first subject and provide to a second subject which has been classified to a similar phenotype 124.

Continuing in reference to FIG. 1, computing device 104 is configured to generate a congenital nourishment program using the at least a nutrition element, wherein the congenital nourishment program includes a consumption model. A “congenital nourishment program,” as used in this disclosure, is a collection of nutrient amounts and nutrition elements 144 for addressing s phenotype 124. Congenital nourishment program 152 may include meals organized into a consumption model. A “consumption model,” as used in this disclosure, is a frequency and magnitude associated with at least a nutrition element 144. A “frequency,” as used in this disclosure, is a number of consumption occurrences associated with a time course, such as daily, weekly, monthly, and the like, of which a nutrition element 144 is intended to be consumed. Frequency may be determined as a function of the identified effect, wherein the frequency of consumption is tailored to provide a sufficient minimal congenital relationship 132 over a period. A “magnitude,” as used in this disclosure, is a serving size of at least a nutrition element 144 as a function of the identified effect. Identifying the magnitude associated with a nutrition element may include calculating a serving size of the at least a nutrition element as a function of the identified effect, where the serving size may be divided into quantities to be consumed according to a frequency. Congenital nourishment program 152 may include gathering, classifying, or otherwise categorizing nutrient amounts and/or nutrition elements 140 lists, which incorporates phenotype-specific recommendations. For instance, nutrition elements 140 may be scored with a numerical score scale that associates a meal, beverage, supplement, and the like, with addressing congenital disorder, alleviating symptoms, and the like. Congenital nourishment program 152 may include selecting nutrition elements 140 according to a threshold score, where items above the threshold are selected and arranged into meals. Threshold score may include a daily threshold, wherein nutrition elements 140 are selected each day according to the threshold; and threshold may include a numerical value relating to symptom prevention, a calculated nutrient amount, among other outputs of system 100 described herein. Determining congenital nourishment program 152 may include machine-learning. For instance and without limitation, training a machine-learning model to identify a scoring rubric for building the congenital nourishment program 152 based on some criteria such as preventing biomarkers from increasing/decreasing, alleviating symptoms, among other criteria. Congenital nourishment program 152 may relate specific phenotype 124 to specific nutrients of interest and provide nutrition element 144 scheduling times and serving sizes for each meal according to the categorization. Congenital nourishment program 152 may differ from one subject to the next according to the magnitude of the disease outline (phenotype 124 and congenital parameter 112).

Continuing in reference to FIG. 1, generating the congenital nourishment program may include receiving a subject preference. A “subject preference”, as used in this disclosure, is a user input that designates a preference related to at least a nutrition element 144. Subject preference may include designations of nutrition elements 140 to avoid and/or include such as particular food groups, ingredients, condiments, spices, dietary restrictions such as ‘no animal products’, cuisine type such as ‘Mediterranean foods’, time of day for eating such as ‘fasting before 10 am’, and the like. Subject preference may include indications of allergies, food intolerances, contraindications associated with medications, and the like, which may represent constraints on curating nutrition elements 140. In this way, computing device 104 may accept an input of subject preference filter, sort, classify, or otherwise modify the data structure of nutrition elements 140 and schedule the nutrition elements 140 into congenital nourishment program 152 in a custom, per-subject manner. Computing device 104 may modify the plurality of nutrition elements 140 as a function of the subject preference, for instance by providing recipes with steps omitted, new steps added, or entirely new recipes altogether utilizing the same or different nutrition elements 140. Computing device 104 may modify the plurality of nutrition elements 140 as a function of the subject preference by generating a new file, based on the preference, and storing and/or retrieving the file from a database, as described in further detail below.

Continuing in reference to FIG. 1, generating the congenital nourishment program 152 may include generating a linear programming function with the plurality of nutrition elements wherein the linear programming function outputs at least an ordering of plurality of nutrition elements according to the congenital relationship 132. An “linear programming function,” as used in this disclosure, is a mathematical objective function that may be used by computing device 104 to score each possible combination of nutrition elements 140, wherein the linear programming function may refer to any mathematical optimization (mathematical programming) to select the ‘best’ element from a set of available alternatives. Selecting the ‘best’ element from a set of available alternatives may include a combination of nutrition elements 140 which achieves the nutrient amounts in addressing congenital disorder in a subject. Alternatively or additionally, linear programming function may generate solutions according to constraints placed by subject preferences.

Still referring to FIG. 1, linear programming function may be formulated as a linear program, which computing device 104 may solve using a linear program, such as without limitation, a mixed-integer program. A “linear program,” as used in this disclosure, is a program that optimizes a linear programming function, given at least a constraint; a linear program may be referred to without limitation as a “linear optimization” process and/or algorithm. For instance, in non-limiting illustrative examples, a given constraint might be a metabolic disorder of a subject, as indicated by Subject preference, and a linear program may use a linear programming function to calculate combinations, considering how these limitations effect combinations. In various embodiments, system 100 may determine a set of instructions towards addressing a subject's congenital parameter 112 that maximizes a total congenital disorder prevention score subject to a constraint that there are other competing objectives. For instance, if achieving one congenital relationship 132 by selecting from a first nutrition element 144 may result in needing to select a second nutrition element 144, wherein each may compete in degradation prevention (e.g. adopting two or more diet types simultaneously may not be feasible, a vegan option and a non-vegan option, and the like). A mathematical solver may be implemented to solve for the set of instructions that maximizes scores; mathematical solver may be implemented on computing device 104 and/or another device in system 100, and/or may be implemented on third-party solver.

Continuing in reference to FIG. 1, a linear programming function may include performing a greedy algorithm process. A “greedy algorithm” is defined as an algorithm that selects locally optimal choices, which may or may not generate a globally optimal solution. For instance, computing device 104 may select combinations of nutrition elements 140 so that values associated therewith are the best value for each category. For instance, in non-limiting illustrative example, optimization may determine the combination of the most efficacious ‘magnitude’, ‘frequency’, ‘nutrition-linked congenital disorder category’, ‘nutrition-linked disorder prevention category’, ‘probiotic’, ‘vegetable’, ‘nutrient amount per meal’, among other categories to provide a combination that may include several locally optimal solutions but may or may not be globally optimal in combination.

With continued reference to FIG. 1, linear programming function may include minimizing a loss function, where a “loss function” is an expression of an output of which a process minimizes to generate an optimal result. For instance, achieving congenital relationships 128 may be set to a nominal value, such as ‘100’, wherein the linear programming function selects elements in combination that reduce the value to ‘0’, wherein the nutrient amounts are ‘100% achieved’. In such an example, ‘maximizing’ would be selecting the combination of nutrition elements 140 that results in achieving nutrient amounts by minimizing the difference. As a non-limiting example, computing device 104 may assign variables relating to a set of parameters, which may correspond to congenital symptom prevention components, calculate an output of mathematical expression using the variables, and select an objective that produces an output having the lowest size, according to a given definition of “size.” Selection of different loss functions may result in identification of different potential combinations as generating minimal outputs, and thus ‘maximizing’ efficacy of the combination.

Continuing in reference to FIG. 1, consumption model 156 may include a nutrition-linked result. A “nutrition-linked result,” is an effect on the subject according to adhering to the consumption model 156 for each nutrition element 144. Nutrition-linked result 160 may include an effect of a nutrient, as described above. Nutrition-linked result 160 may include an indication of whether a congenital symptom may be addressed by a nutrition element. Nutrition-linked result 160 may include any information regarding the consumption model 156 as it relates to congenital factor 108 and/or congenital parameter 112, for instance if a biomarker may be affected, a symptom may be exacerbated, and the like.

Continuing in reference to FIG. 1, computing device 104 may use calculated nutrient amounts from nutraceutical model 136 to determine nutrition elements 140 more precisely. For instance, computing device 104 may retrieve a variety of nutrition elements 140 which contain particular vitamins, minerals, anti-inflammatory molecules, phytonutrients, antioxidants, bioactive molecules, and the like, which do not violate any other congenital symptom prevention information associated with congenital parameter 112. Computing device 104 may mix-and-match nutrition elements 140 to arrive at a particular calorie amount, range of calories, or number of macromolecules, while achieving nutrient amounts. In this way, de novo nutrition element 144 that may not exist may be created from various ingredients according to their nutrient profile.

Continuing in reference to FIG. 1, generating the congenital nourishment program 152 may include generating a congenital nourishment program classifier using a congenital nourishment program classification machine-learning process to classify nutrition elements 140 according to congenital symptom and/or disorder, and outputting a plurality of nutrition elements as a function of the congenital nourishment program classifier. Congenital nourishment program classifier may include any classifier, as described herein, generated by a classification machine-learning process using training data, performed by a machine-learning module as described in further detail below. Training data for congenital nourishment program classifier may include sets of data entries that include nutrition elements 140 that are correlated to phenotype 124 relating to symptoms, biomarkers, and disorders. Classifier may be trained to automatedly locate, sort, and output nutrition elements 140 according to calculated nutrient amounts for the subject belonging to such a categorization. Such training data may originate via a database, the Internet, research repository, and the like, as described herein for training data for other machine-learning processes. Training data may include foods, supplements, probiotics, nutraceuticals, and the like, correlated to nutrition facts, medicinal qualities, and the like, which a classifier may be trained to identify relationships that aid in sorting nutrition elements 140 according to a relationship to a disorder. Congenital nourishment program classifier may accept an input of phenotype 124 and output a plurality of nutrition elements 140 with associated consumption model according to relationships identified in training data. For instance and without limitation, congenital nourishment program classifier may identify relationships between individual fruits and vegetables, that when more vegetables are selected, certain fruits may not be necessary to schedule within the same timeframe. Such a classification process may determine a function, system of equations, and the like, which can be solved for in determining which nutrition elements 140 are useful toward obtaining the nutrient amounts, while not missing some lower limits of nutrient amounts (trace elements) and not exceeding upper limits for other nutrient amounts (calories).

Continuing in reference to FIG. 1, congenital nourishment program 152 may include a nourishment score. A “nourishment score,” as used in this disclosure, reflects the level of subject participation in the congenital nourishment program 152 and the level of congenital disorder in the subject as a function of adherence to congenital nourishment program 152. Nourishment score 164 may include a numerical value, metric, parameter, and the like, described by a function, vector, matrix, or any other mathematical arrangement. Nourishment score 164 may include enumerating a subject's current nourishment as it relates to symptoms alleviation, increased/decrease biomarker levels and/or concentrations, and/or congenital disorder prevention. Generating nourishment score 164 may include using a machine-learning process, algorithm, and/or model to derive a numerical scale along which to provide a numerical value according to a subject's congenital parameter 112 and participation in congenital nourishment program 152 generated from congenital parameter 112. For instance, such a machine-learning model may be trained with training data, wherein training data contains data entries of nutrient amounts correlated to congenital disorder prevention. Such a machine-learning model may be trained with said training data to be used by computing device 104 to correlate the consumption of particular nutrition elements 140 in congenital nourishment program 152 to achieving some congenital relationship 132, and how congenital relationship 132 relates to symptom alleviation, and the like. Training data for a machine-learning model for generating nourishment score 164 may include a plurality of data entries including nutrient amounts correlated to effects on phenotype 124, wherein the trained model may accept inputs of nutritional input from subject and automatedly determine how the score should increase and/or decrease based on the nutrient targets for the subject. Such training data may originate from any source as descried above, such as a database, web browser and the Internet, physician, peer-reviewed research, and the like.

Continuing in reference to FIG. 1, in non-limiting illustrating examples, falling short of copper and B-complex vitamin nutrient amounts, may have a particular effect on nourishment score 164 for an individual who has been classified to a certain phenotype 124. Where, chronically falling short of the nutrient amount results in a (−3) score each month but falling within the nutrient amount range affords (+1) score for each month; the target amount for the preceding month may dictate the score change for each subsequent month. In such a case, a machine-learning model may derive an algorithm which dictates the amount to increase/decrease nourishment score 164 for that particular phenotype 124 according to the nutrient amounts. In this case, the machine-learning model is trained to identify the relationship between nutrient amounts and effect on score to derive an equation that relates scoring criteria to nutritional input. The score is then calculated using the model and nutritional input from the subject. In this way, computing device 104 may calculate a nourishment score 164 as a function of a subject's participation in Congenital nourishment program 152, where nourishment score 164 is updated with each nutrition element 144 consumed by subject.

Continuing in reference to FIG. 1, in an embodiment and without limitation, classification of a subject to a congenital disorder category as a function of their congenital factor 108 data may include a predictive diagnosis pertaining to the subject. This may include: 1) diagnosis based on comparison to a subject cohort, and/or 2) diagnosis based on comparison to normalized thresholds, as the cohort may be susceptible to undiagnosed disorders. This may include: 1) retrieving a congenital parameter 112, 2) determining a threshold value, 3) comparing the congenital parameter 112 to the threshold value, and 4) classifying subject to a phenotype 124 (congenital disorder category) as a function of the comparison. In this instance, threshold value may include a calculated numerical relating to a cohort of subjects, classified based on numerous criteria, which a subject's congenital parameter may be compared to determine the presence of a congenital issue, including secondary issues which may be tangentially related to a current diagnosis. The classification of subject to congenital disorder category may be used to derive congenital relationships 132. The congenital relationships 132 may be calculated as a function of relationships between biomarkers summarized in the congenital parameter and effects of nutrients on the biomarkers as it contributes to the congenital disorder category. Congenital relationships 132 may then be used as an input to generate an output of food recommendations (may include querying via the Internet for nutrition elements and classification by phenotype 124). Nutrition recommendations may be assigned a score relating to their effect on the congenital disorder the subject was categorized to. Nutrition elements may be arranged using a linear programming function into daily, weekly, monthly consumption models 156 focused around addressing the congenital disorder according to constraints arising from user input. Addressing congenital disorder with nutrition may include prevention of future congenital disorder, reducing risk, and the like.

Continuing in reference to FIG. 1, a differentiating factor may be found in the concept that a compendium of subject data may be used to classify subjects according to subsets of subjects based on biological extraction data. Subjects may be assigned predetermined diets or custom, de novo diets that are generated based on two factors: 1) assigning subjects to diet types and/or nutrient amounts according to how they classify to cohorts of alike subjects. For instance, if the subject's data indicates a particular predisposition to nutritional surpluses, deficiencies, and the like, the subject may be assigned to what alike subjects have been assigned. And 2) outcomes associated with subject data. For instance, subjects with insulin insensitivity may have improved health outcomes associated with a particular congenital relationship which was found effective in controlling blood glucose levels. The subject may be assigned a custom diet focused on this paradigm as a result of their congenital factor 108 data relating to that subset of subjects. In this case, a variable may be assigned to each element of biological extraction data for each subject where such a variable is associated with some outcome relating to nutrient amounts. This process may be performed with machine-learning processes as described, without limitation, in U.S. Nonprovisional application Ser. No. 17/106,588, filed Nov. 30, 2020, titled “METHODS AND SYSTEMS FOR DETERMINING A PREDICTIVE INTERVENTION USING BIOMARKERS,” the entirety of which is incorporated herein by reference.

Continuing in reference to FIG. 1, machine-learning may identify relationships in training data (subsets of subject data—biological extraction and nutritional input data) which may be assigned variables to derive an algorithm, function, equation, and the like, which describes a congenital relationship. Variables may be assigned values based on thresholds relating to the biomarker levels, for instance as found among cohorts of healthy adults. This way, population-based nutrigenomics may be employed to individual subjects who may be classified to consumption models based on how they compare to entire populations of subjects. Such populations of subjects may be subsets that belong in a “goal category” relating to the subject. For instance, if the subject has a BMI of 35 and has a target goal of a BMI of 15-20, the target population used may be subjects within the goal BMI, including their corresponding biological extraction variables. The system may then calculate nutrient amounts and identify nutrition elements that will result in the subject's BMI reaching the goal population. This may include a nutrient-biomarker relationship (congenital relationship) to be derived among the population the subject belongs and the target population. Comparison between the population may identify biomarkers that are to address and potentially differences in diet that may address the difference in population biomarker.

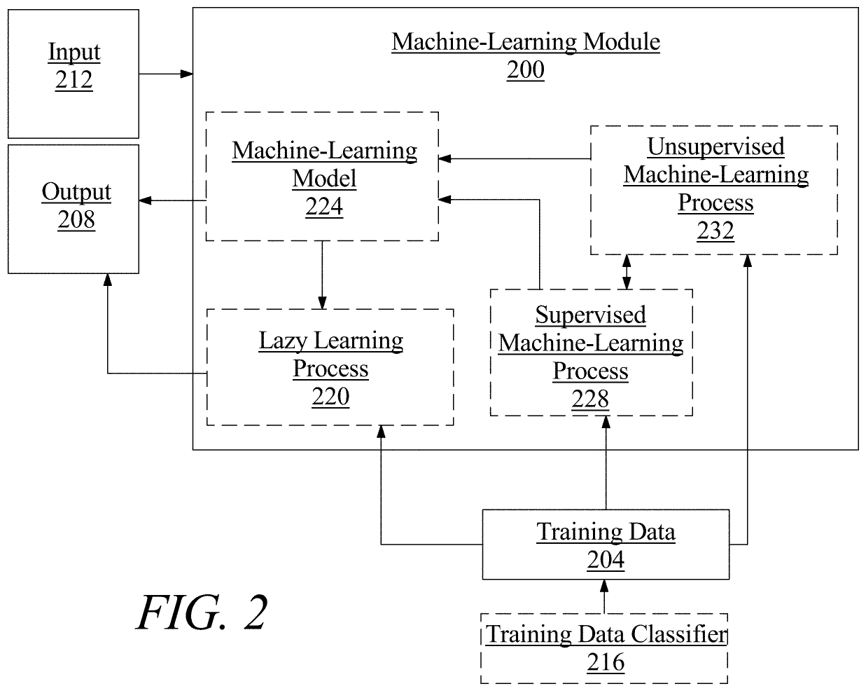

Referring now to FIG. 2, an exemplary embodiment of a machine-learning module 200 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 204 to generate an algorithm that will be performed by a computing device/module to produce outputs 208 given data provided as inputs 212; this is in contrast to a non-machine learning software program where the commands to be executed are determined in advance by a subject and written in a programming language.

Still referring to FIG. 2, “training data,” as used herein, is data containing correlations that a machine-learning process may use to model relationships between two or more categories of data elements. For instance, and without limitation, training data 204 may include a plurality of data entries, each entry representing a set of data elements that were recorded, received, and/or generated together; data elements may be correlated by shared existence in a given data entry, by proximity in a given data entry, or the like. Multiple data entries in training data 204 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 204 according to various correlations; correlations may indicate causative and/or predictive links between categories of data elements, which may be modeled as relationships such as mathematical relationships by machine-learning processes as described in further detail below. Training data 204 may be formatted and/or organized by categories of data elements, for instance by associating data elements with one or more descriptors corresponding to categories of data elements. As a non-limiting example, training data 204 may include data entered in standardized forms by persons or processes, such that entry of a given data element in a given field in a form may be mapped to one or more descriptors of categories. Elements in training data 204 may be linked to descriptors of categories by tags, tokens, or other data elements; for instance, and without limitation, training data 204 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 2, training data 204 may include one or more elements that are not categorized; that is, training data 204 may not be formatted or contain descriptors for some elements of data. Machine-learning algorithms and/or other processes may sort training data 204 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 204 to be made applicable for two or more distinct machine-learning algorithms as described in further detail herein. Training data 204 used by machine-learning module 200 may correlate any input data as described in this disclosure to any output data as described in this disclosure.

Further referring to FIG. 2, training data may be filtered, sorted, and/or selected using one or more supervised and/or unsupervised machine-learning processes and/or models as described in further detail herein; such models may include without limitation a training data classifier 216. Training data classifier 216 may include a “classifier,” which as used in this disclosure is a machine-learning model as defined herein, such as a mathematical model, neural net, or program generated by a machine learning algorithm known as a “classification algorithm,” as described in further detail herein, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 200 may generate a classifier using a classification algorithm, defined as a process whereby a computing device and/or any module and/or component operating thereon derives a classifier from training data 204. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 216 may classify elements of training data to elements that characterizes a sub-population, such as a subset of congenital factor 108 and/or other analyzed items and/or phenomena for which a subset of training data may be selected.

Still referring to FIG. 2, machine-learning module 200 may be configured to perform a lazy-learning process 220 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of predictions may be performed to cover an initial heuristic and/or “first guess” at an output and/or relationship. As a non-limiting example, an initial heuristic may include a ranking of associations between inputs and elements of training data 204. Heuristic may include selecting some number of highest-ranking associations and/or training data 204 elements, such as classifying congenital factor 108 elements to congenital parameter 112 elements and assigning a value as a function of some ranking association between elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail herein.

Alternatively or additionally, and with continued reference to FIG. 2, machine-learning processes as described in this disclosure may be used to generate machine-learning models 224. A “machine-learning model,” as used in this disclosure, is a mathematical and/or algorithmic representation of a relationship between inputs and outputs, as generated using any machine-learning process including without limitation any process as described above and stored in memory; an input is submitted to a machine-learning model 224 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 224 may be generated by creating an artificial neural network, such as a convolutional neural network comprising an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. A machine-learning model may be used to derive numerical scales for providing numerical values to congenital parameter 112 and/or nourishment score 164, and the like, as described above, to “learn” the upper and lower limits to the numerical scale, the increments to providing scoring, and the criteria for increasing and decreasing elements encompassed in the congenital parameter 112 and/or nourishment score 164, and the like. A machine-learning model may be used to “learn” which elements of congenital factor 108 have what effect on congenital parameter 112, and which elements of congenital parameter 112 are affected by particular nutrition elements 140 and the magnitude of effect, and the like. The magnitude of the effect may be enumerated and provided as part of system 100, where nutrition elements 140 are communicated to subject for their symptom alleviation properties.

Still referring to FIG. 2, machine-learning algorithms may include at least a supervised machine-learning process 228. At least a supervised machine-learning process 228, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include a congenital parameter 112 (potentially classified into congenital disorder categories 120), as described above as inputs, nutrition element 144 outputs, and a ranking function representing a desired form of relationship to be detected between inputs and outputs; ranking function may, for instance, seek to maximize the probability that a given input (such as nutrient amounts) and/or combination of inputs is associated with a given output (congenital nourishment program 152 that incorporate nutrient elements 120 to achieve nutrient amounts that are ‘best’ for phenotype 124) to minimize the probability that a given input is not associated with a given output, for instance finding the most frequency, magnitude, and what the nutrition elements 140 should be, and the like. Ranking function may be expressed as a risk function representing an “expected loss” of an algorithm relating inputs to outputs, where loss is computed as an error function representing a degree to which a prediction generated by the relation is incorrect when compared to a given input-output pair provided in training data 204. Persons skilled in the art, upon the benefit of reviewing the entirety of this disclosure, will be aware of various possible variations of at least a supervised machine-learning process 228 that may be used to determine relation between inputs and outputs. Supervised machine-learning processes may include classification algorithms as defined above.

Further referring to FIG. 2, machine learning processes may include at least an unsupervised machine-learning process 232. An unsupervised machine-learning process 232, as used herein, is a process that derives inferences in datasets without regard to labels; as a result, an unsupervised machine-learning process 232 may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 2, machine-learning module 200 may be designed and configured to create a machine-learning model 224 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 2, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

Still referring to FIG. 2, models may be generated using alternative or additional artificial intelligence methods, including without limitation by creating an artificial neural network, such as a convolutional neural network comprising an input layer of nodes, one or more intermediate layers, and an output layer of nodes. Connections between nodes may be created via the process of “training” the network, in which elements from a training data 204 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. This network may be trained using training data 204.

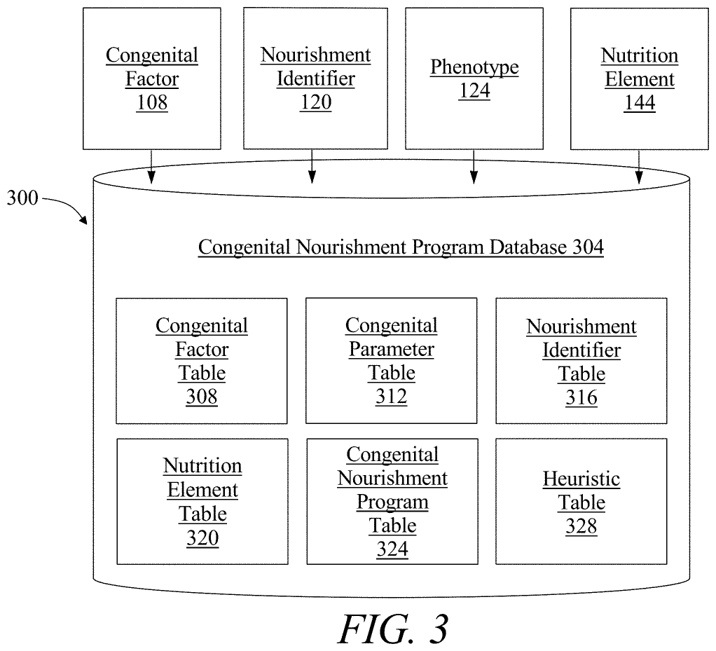

Referring now to FIG. 3, a non-limiting exemplary embodiment 300 of a congenital nourishment program database 304 is illustrated. Congenital factor(s) 108 from a plurality of subjects, for instance for generating a training data classifier 216, may be stored and/or retrieved in congenital nourishment program database 304. Congenital factor(s) 108 data from a plurality of subjects for generating training data 204 may also be stored and/or retrieved from a congenital nourishment program database 304. Computing device 104 may receive, store, and/or retrieve training data 204, wearable device data, physiological sensor data, biological extraction data, and the like, from congenital nourishment program database 304. Computing device 104 may store and/or retrieve nutrient machine-learning model 116, congenital classifier 128, among other determinations, I/O data, models, and the like, from congenital nourishment program database 304.

Continuing in reference to FIG. 3, congenital nourishment program database 304 may be implemented, without limitation, as a relational database, a key-value retrieval database such as a NOSQL database, or any other format or structure for use as a database that a person skilled in the art would recognize as suitable upon review of the entirety of this disclosure. Congenital nourishment program database 304 may alternatively or additionally be implemented using a distributed data storage protocol and/or data structure, such as a distributed hash table and the like. Congenital nourishment program database 304 may include a plurality of data entries and/or records, as described above. Data entries in a congenital nourishment program database 304 may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistent with this disclosure.

Further referring to FIG. 3, congenital nourishment program database 304 may include, without limitation, congenital factor table 308, congenital parameter table 312, nourishment identifier table 316, nutrition element table 320, congenital nourishment program table 324, and/or heuristic table 328. Determinations by a machine-learning process, machine-learning model, ranking function, and/or classifier, may also be stored and/or retrieved from the congenital nourishment program database 304. As a non-limiting example, congenital nourishment program database 304 may organize data according to one or more instruction tables. One or more congenital nourishment program database 304 tables may be linked to one another by, for instance in a non-limiting example, common column values. For instance, a common column between two tables of congenital nourishment program database 304 may include an identifier of a submission, such as a form entry, textual submission, accessory device tokens, local access addresses, metrics, and the like, for instance as defined herein; as a result, a search by a computing device 104 may be able to retrieve all rows from any table pertaining to a given submission or set thereof. Other columns may include any other category usable for organization or subdivision of data, including types of data, names and/or identifiers of individuals submitting the data, times of submission, and the like; persons skilled in the art, upon reviewing the entirety of this disclosure, will be aware of various ways in which data from one or more tables may be linked and/or related to data in one or more other tables.

Continuing in reference to FIG. 3, in a non-limiting embodiment, one or more tables of a congenital nourishment program database 304 may include, as a non-limiting example, a congenital factor table 308, which may include categorized identifying data, as described above, including congenital factor 108 data such as genetic data, epigenetic data, microbiome data, physiological data, biological extraction data, and the like. Congenital factor table 308 may include congenital factor 108 categories according to gene expression patterns, SNPs, mutations, enzyme specific activity and concentration, phosphorylation data, blood biomarker data, data concerning metabolism of nutrition elements 140, pharmacokinetics, nutrient absorption, and the like, and may include linked tables to mathematical expressions that describe the impact of each congenital factor 108 datum on congenital parameter 112, for instance threshold values for gene expression levels of biomarkers, blood protein concentrations, WBC counts, and the like, as it relates to congenital parameters, phenotype 124, and the like. One or more tables may include congenital parameter table 312, which may include data regarding congenital factor 108, thresholds, scores, metrics, values, categorizations, and the like, that system 100 may use to calculate, derive, filter, retrieve and/or store current symptoms, formulas relating biomarkers to congenital parameters, biomarkers as they relate to phenotype 124, and the like. One or more tables may include nourishment identifier table 316, which may include data on nutrition elements 140 for instance classified to phenotype 124, classified to data from alike subjects with similar congenital factor 108, congenital parameter 112, and the like, that system 100 may use to calculate, derive, filter, retrieve and/or store nutrition elements 140. One or more tables may include nutrition element table 320, which may include functions, model, equations, algorithms, and the like, using to calculate or derive congenital relationship 132 relating to congenital parameter 112 and/or phenotype 124, may include nutrient amounts organized by nutrient, nutrient classification, subject data such as age, sex, symptom severity, and the like. One of more tables may include a congenital nourishment program table 324, which may include nutrition element 144 identifiers, consumption models 156, times associated with nutrition elements 140 regarding times to eat, identifiers of meals, recipes, ingredients, frequency, magnitude, diet types, and the like. One or more tables may include, without limitation, a heuristic table 328, which may organize rankings, scores, models, outcomes, functions, numerical values, scales, arrays, matrices, congenital relationships 132, and the like, that represent determinations, probabilities, metrics, parameters, values, and the like, include one or more inputs describing potential mathematical relationships, as described herein.

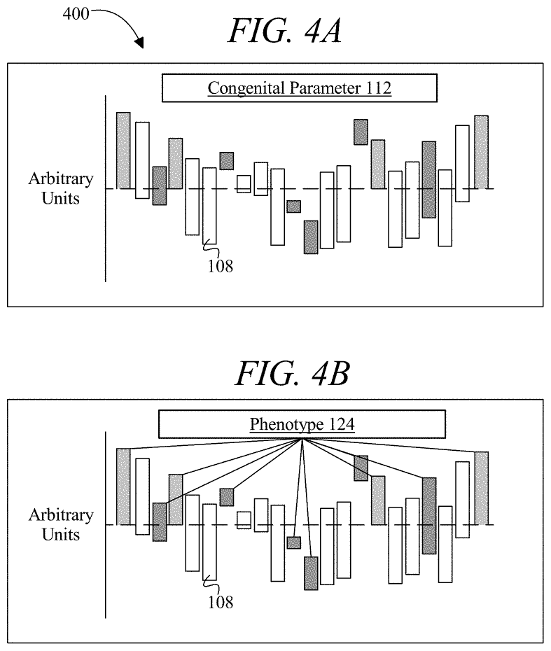

Referring now to FIGS. 4A and 4B, a non-limiting exemplary embodiment 400 of congenital parameters 112 is illustrated. Congenital parameter 112 may include a variety of congenital factor 108 categories, for instance 22 distinct categories, as shown in FIGS. 4A and 4B. Each congenital factor 108 may be assigned a value, such as an arbitrary value, where some congenital factor 108, such as those shaded in light grey, may relate to absolute scales from [0, x], where x is a maximal value and the range of values for the congenital factor 108 cannot be below a ‘zero amount’. Some congenital factor 108, such as those shaded in dark grey, may relate to gene expression levels, wherein, the congenital factor 108 is enumerated as a ‘box plot’ that illustrates the range of concentrations of proteins, blood cell counts, hormone levels, and the like, in a population of subjects organized according to, for instance age, fitness level, nutrition, and the like. In such an example, the dashed line may relate to a ‘normal threshold’ above which the biomarker is considered elevated, below which is decreased. Each congenital factor 108 may have associated with it a numerical score, or some other identifying mathematical value that computing device 104 may assign. Persons skilled in the art, upon the benefit of this disclosure in its entirety, may appreciate that for each subject, any number of congenital factor 108 may be enumerated and assigned a value according to congenital parameter machine-learning model 116 and the breadth of biomarker data provided. Congenital parameter 112 may be graphed, or otherwise displayed, according to the enumeration by congenital parameter machine-learning model 116. Each bar of the bar graph, or combinations of bar graph categories, may instruct a classification of a subject's congenital parameter 112 to a phenotype 124.

Still referring now to FIGS. 4A and 4B, in non-limiting exemplary illustrations congenital parameter 112 may be classified to a phenotype 124. Some and/or all of the congenital factor 108 summarized in congenital parameter 112 may be used to classify an individual to a particular phenotype 124. For instance, as shown in FIG. 4B, ten of the 22 congenital factor 108 categories may be used to classify congenital parameter 112 to one or more phenotypes 124. Alternatively or additionally, congenital parameter machine-learning model 116 may be trained to assign congenital factor 108 to a phenotype 124, wherein computing device 104 may know the identity of phenotype 124 according to which phenotype 124 has the most identifying data points. Alternatively or additionally, congenital classifier 128 may be trained to assign subject to a phenotype 124 according to patterns observed in congenital parameter 112, for instance according to data from a subset of subjects.

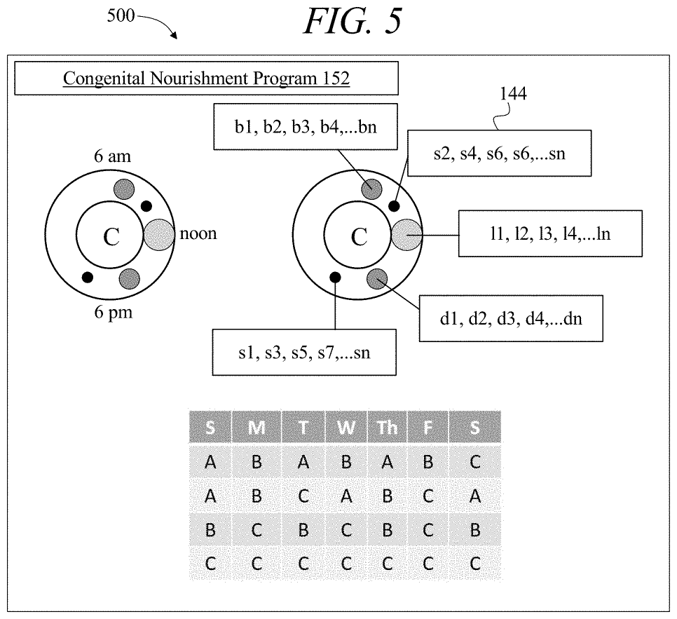

Referring now to FIG. 5, a non-limiting exemplary embodiment 500 of a congenital nourishment program 152 is illustrated. Congenital nourishment program 152 may include a schedule for arranging nutrition elements 140, according to for instance a 24-hour timetable, as designated on the left, where consumption is planned along a subject's typical day-night cycle, beginning at ˜6 am until just after 6 pm. Nutrition element 144 may include breakfast (denoted as mid-sized dark grey circle), which may correspond to a file of breakfast-related plurality of nutrition elements 140 (denoted b1, b2, b3, b4 . . . bn, to the nth breakfast item). Nutrition element 144 may include snacks eaten throughout the day to, for instance achieve nutrient amounts missing from meals (denoted as small black circles), which may correspond to a file of snacking-related plurality of nutrition elements 140 (denoted s1, s2, s3, s4 . . . sn, to the nth snacking item). Nutrition element 144 may include dinner (denoted as large-sized light grey circle), which may correspond to a file of dinner-related plurality of nutrition elements 140 (denoted d1, d2, d3, d4 . . . dn, to the nth dinner item). Congenital nourishment program 152 may include a variety of custom diets, as denoted in the monthly schedule at the bottom, Sunday through Saturday. Congenital nourishment program 152 ‘C’ is shown, which may be an idealistic goal for subject to achieve by the end of the month, where nourishment plan ‘A’ and ‘B’ are intermediate plans intended to wean subject to the ‘ideal’ plan. Nutrition elements 140 classified by category and may be further modified by ‘A’, ‘B’, ‘C’, and the like, according to subject preferences input into computing device 104. Circle sizes, denoting nutrition element 144 classes may relate to magnitude, which are graphed along the circle corresponding to the frequency they are expected to be consumed. Subject may indicate which nutrition element 144 from each category was consumed, and when it was consumed, to arrive at nourishment score 164.

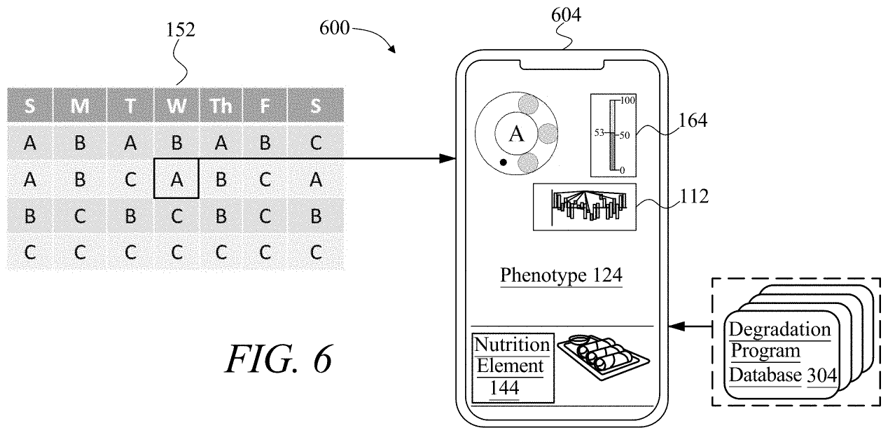

Referring now to FIG. 6, a non-limiting exemplary embodiment 600 of a user device 604 is illustrated. User device 604 may include computing device 104, a “smartphone,” cellular mobile phone, desktop computer, laptop, tablet computer, internet-of-things (JOT) device, wearable device, among other devices. User device 604 may include any device that is capable of communicating with computing device 104, congenital nourishment program database 304, or able to receive, transmit, and/or display, via a graphical user interface, congenital parameter 112, phenotype 124, nutrition element 144, congenital nourishment program 152, nourishment score 164, among other outputs from system 100. User device 604 may provide a congenital parameter 112, for instance as a collection of metrics determined from congenital factor 108 data. User device 604 may provide phenotype 124 that was determined as a function of congenital classifier 128 and congenital parameter 112. User device 604 may provide data concerning nutrient amounts, including the levels of specific nutrients, nutrient ranges, nutrients to avoid (for instance, if it exacerbates symptoms), and the like. User device 604 may link timing of foods to preemptive ordering interface for ordering a nutrition element 144, for instance and without limitation, through a designated mobile application, mapping tool or application, and the like, and a radial search method about a subject's current location as described in U.S. Nonprovisional application Ser. No. 17/087,745, filed Nov. 3, 2020, titled “A METHOD FOR AND SYSTEM FOR PREDICTING ALIMENTARY ELEMENT ORDERING BASED ON BIOLOGICAL EXTRACTION,” the entirety of which is incorporated herein by reference. User device 604 may display nutrient elements 120 as a function of location, for instance and without limitation, as described in User device 604 may link nourishment consumption model 120 to a scheduling application, such as a ‘calendar’ feature on subject device, which may set audio-visual notifications, timers, alarms, and the like.

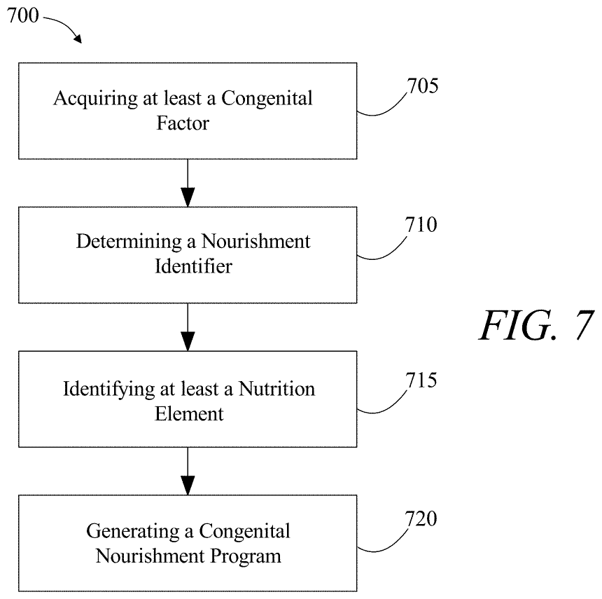

Referring now to FIG. 7, an exemplary embodiment 700 of a method for generating a congenital nourishment program 152 for addressing congenital disorders is illustrated. At step 705, the method including acquiring, by a computing device 104, at least a congenital factor 108 relating to a subject; this may be implemented, without limitation, as described above in FIGS. 1-6.

Continuing in reference to FIG. 7, at step 710, method includes determining, by the computing device 104, using the congenital factor 108, a nourishment identifier 120, wherein generating the nourishment identifier 120 includes identifying, using the congenital factor 108, a phenotype 124, generating, using the phenotype 124, a congenital relationship 132, wherein the congenital relationship 132 relates at least an effect of at least a nourishment identifier 120 on the phenotype 124 and determining the nourishment identifier 120 as a function of the at least an effect. Determining the nourishment identifier may include training a parameter machine-learning model with training data that includes a plurality of data entries correlating congenital factors to a plurality of congenital parameters, generating the congenital parameter as a function of the parameter machine-learning model and the at least a congenital factor, and determining the nourishment identifier as a function of the congenital parameter. Identifying the phenotype 124 may include training a congenital classifier 128 using training data which includes a plurality of data entries of congenital factors 108 from a subset of categorized subjects, classifying the congenital factor 108 to the phenotype 124 using the congenital classifier 128 and identifying the phenotype 124 as a function of the classifying. Generating the congenital relationship 132 may include generating a nutraceutical model 136 using a machine-learning process and training data which includes a plurality of data entries correlating effects of nourishment identifiers 120 to phenotypes 124 and determining the congenital relationship 132 as a function of the nutraceutical model 136 and the phenotype 124. Computing device 104 may be further configured to generate a nourishment training dataset 140 from a plurality of congenital relationship 132 outputs of the nutraceutical model 136; this may be implemented, without limitation, as described above in FIGS. 1-6.

Continuing in reference to FIG. 7, at step 715, method includes identifying, by the computing device 104, using the nourishment identifier 120, at least a nutrition element 144. Identifying the at least a nutrition element 144 may include generating a nutrition model 148 using training data including a plurality of data entries of nourishment identifiers 120 correlating to nutrition elements 144 and determining the at least a nutrition element 144 as a function of the nutrition model 140 and the nourishment identifier 120. Identifying at least a nutrition element 144 may include retrieving a plurality of nutrition elements 144 from a data repository as a function of the at least a congenital relationship 132; and; this may be implemented, without limitation, as described above in FIGS. 1-6.

Continuing in reference to FIG. 7, at step 720, method includes generating, by the computing device 104, a congenital nourishment program 152 using the at least a nutrition element 144, wherein the congenital nourishment program 152 includes a consumption model 156. Generating the congenital nourishment program 152 may include generating a linear programming function with the plurality of nutrition elements wherein the linear programming function outputs at least an ordering of the plurality of nutrition elements according to the consumption model 156. Consumption model 156 may include a nutrition-linked result 160. Congenital nourishment program 152 may include a nourishment score 164; this may be implemented, without limitation, as described above in FIGS. 1-6.

It is to be noted that any one or more of the aspects and embodiments described herein may be conveniently implemented using one or more machines (e.g., one or more computing devices that are utilized as a subject computing device for an electronic document, one or more server devices, such as a document server, and the like) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methods and/or embodiments described herein. Examples of a machine-readable storage medium include, but are not limited to, a magnetic disk, an optical disc (e.g., CD, CD-R, DVD, DVD-R, and the like), a magneto-optical disk, a read-only memory “ROM” device, a random-access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, and the like), a web appliance, a network router, a network switch, a network bridge, any machine capable of executing a sequence of instructions that specify an action to be taken by that machine, and any combinations thereof. In one example, a computing device may include and/or be included in a kiosk.

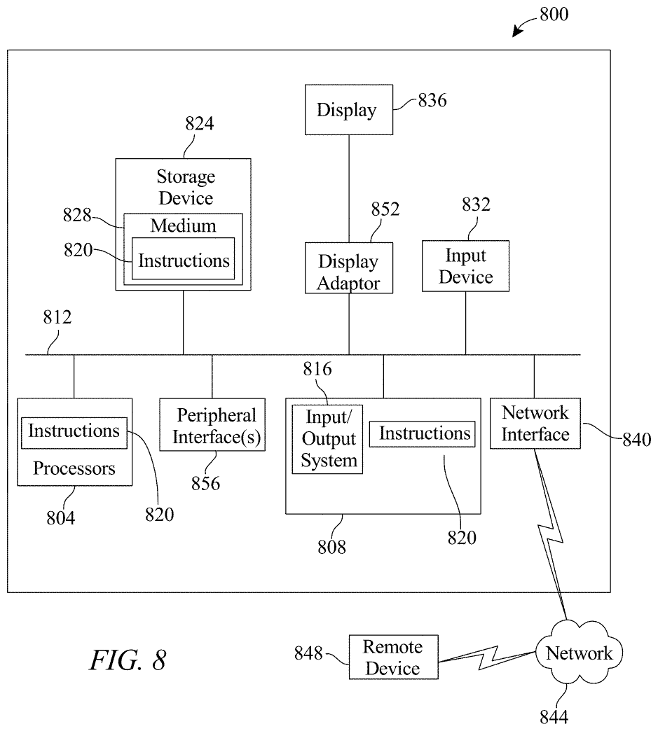

FIG. 8 shows a diagrammatic representation of one embodiment of a computing device in the exemplary form of a computer system 800 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 800 includes a processor 804 and a memory 808 that communicate with each other, and with other components, via a bus 812. Bus 812 may include any of several types of bus structures including, but not limited to, a memory bus, a memory controller, a peripheral bus, a local bus, and any combinations thereof, using any of a variety of bus architectures.

Processor 804 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 804 may be organized according to Von Neumann and/or Harvard architecture as a non-limiting example. Processor 804 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating-point unit (FPU), and/or system on a chip (SoC)

Memory 808 may include various components (e.g., machine-readable media) including, but not limited to, a random-access memory component, a read only component, and any combinations thereof. In one example, a basic input/output system 816 (BIOS), including basic routines that help to transfer information between elements within computer system 800, such as during start-up, may be stored in memory 808. Memory 808 may also include (e.g., stored on one or more machine-readable media) instructions (e.g., software) 820 embodying any one or more of the aspects and/or methodologies of the present disclosure. In another example, memory 808 may further include any number of program modules including, but not limited to, an operating system, one or more application programs, other program modules, program data, and any combinations thereof.

Computer system 800 may also include a storage device 824. Examples of a storage device (e.g., storage device 824) include, but are not limited to, a hard disk drive, a magnetic disk drive, an optical disc drive in combination with an optical medium, a solid-state memory device, and any combinations thereof. Storage device 824 may be connected to bus 812 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 824 (or one or more components thereof) may be removably interfaced with computer system 800 (e.g., via an external port connector (not shown)). Particularly, storage device 824 and an associated machine-readable medium 828 may provide nonvolatile and/or volatile storage of machine-readable instructions, data structures, program modules, and/or other data for computer system 800. In one example, software 820 may reside, completely or partially, within machine-readable medium 828. In another example, software 820 may reside, completely or partially, within processor 804.

Computer system 800 may also include an input device 832. In one example, a subject of computer system 800 may enter commands and/or other information into computer system 800 via input device 832. Examples of an input device 832 include, but are not limited to, an alpha-numeric input device (e.g., a keyboard), a pointing device, a joystick, a gamepad, an audio input device (e.g., a microphone, a voice response system, and the like), a cursor control device (e.g., a mouse), a touchpad, an optical scanner, a video capture device (e.g., a still camera, a video camera), a touchscreen, and any combinations thereof. Input device 832 may be interfaced to bus 812 via any of a variety of interfaces (not shown) including, but not limited to, a serial interface, a parallel interface, a game port, a USB interface, a FIREWIRE interface, a direct interface to bus 812, and any combinations thereof. Input device 832 may include a touch screen interface that may be a part of or separate from display 836, discussed further below. Input device 832 may be utilized as a subject selection device for selecting one or more graphical representations in a graphical interface as described above.

A subject may also input commands and/or other information to computer system 800 via storage device 824 (e.g., a removable disk drive, a flash drive, and the like) and/or network interface device 840. A network interface device, such as network interface device 840, may be utilized for connecting computer system 800 to one or more of a variety of networks, such as network 844, and one or more remote devices 848 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus, or other relatively small geographic space), a telephone network, a data network associated with a telephone/voice provider (e.g., a mobile communications provider data and/or voice network), a direct connection between two computing devices, and any combinations thereof. A network, such as network 844, may employ a wired and/or a wireless mode of communication. In general, any network topology may be used. Information (e.g., data, software 820, and the like) may be communicated to and/or from computer system 800 via network interface device 840.

Computer system 800 may further include a video display adapter 852 for communicating a displayable image to a display device, such as display device 836. Examples of a display device include, but are not limited to, a liquid crystal display (LCD), a cathode ray tube (CRT), a plasma display, a light emitting diode (LED) display, and any combinations thereof. Display adapter 852 and display device 836 may be utilized in combination with processor 804 to provide graphical representations of aspects of the present disclosure. In addition to a display device, computer system 800 may include one or more other peripheral output devices including, but not limited to, an audio speaker, a printer, and any combinations thereof. Such peripheral output devices may be connected to bus 812 via a peripheral interface 856. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1. A system for generating a nourishment program for addressing congenital disorders, the system comprising:
a computing device, wherein the computing device is configured to:
acquire at least a congenital factor relating to a subject;
determine, using the at least a congenital factor, a nourishment identifier, wherein generating the nourishment identifier includes:
identifying, using the at least a congenital factor, a phenotype;
generating, using the phenotype, a congenital relationship, wherein the congenital relationship relates at least an effect of the nourishment identifier on the phenotype; and
determining the nourishment identifier as a function of the congenital relationship;
identify, using the nourishment identifier, at least a nutrition element; and
generate a consumption model based on the at least a nutrition element.

2. The system of claim 1, wherein determining the nourishment identifier further comprises:
training a parameter machine-learning model with training data that includes a plurality of data entries correlating congenital factors to congenital parameters; and
generating the congenital parameter as a function of the parameter machine-learning model and the at least a congenital factor; and
determining the nourishment identifier as a function of the congenital parameter.

3. The system of claim 1, wherein identifying the phenotype further comprises:
training a congenital classifier using training data which includes a plurality of data entries of congenital factors from a subset of categorized subjects;
classifying the congenital factor to the phenotype using the congenital classifier; and
identifying the phenotype as a function of the classifying.

4. The system of claim 3, wherein classifying further comprises classifying the congenital parameter to a nutrition-linked congenital disorder category.

5. The system of claim 1, wherein generating the congenital relationship further comprises:
generating a nutraceutical model using a machine-learning process and training data which includes a plurality of data entries correlating effects of nourishment identifiers to phenotypes; and
determining the congenital relationship as a function of the nutraceutical model and the phenotype.

6. The system of claim 4, wherein computing device is further configured to generate a nourishment training dataset from a plurality of congenital relationship outputs of the nutraceutical model.

7. The system of claim 1, wherein identifying the at least a nutrition element further comprises:
generating a nutrition model using training data including a plurality of data entries of nourishment identifiers correlating to nutrition elements; and
determining the at least a nutrition element as a function of the nutrition model and the nourishment identifier.

8. The system of claim 7, wherein generating the congenital nourishment program further comprises generating a linear programming function with the plurality of nutrition elements wherein the linear programming function outputs at least an ordering of the plurality of nutrition elements according to the consumption model.

9. The system of claim 1, where the consumption model comprises a nutrition-linked result.

10. The system of claim 1, wherein the congenital nourishment program includes a nourishment score.

11. A method for generating a congenital nourishment program for addressing congenital disorders, the method comprising:
acquiring, by the computing device, at least a congenital factor relating to a subject;
determining, by the computing device, using the at least a congenital factor, a nourishment identifier, wherein generating the nourishment identifier includes:
identifying, using the at least a congenital factor, a phenotype;
generating, using the phenotype, a congenital relationship, wherein the congenital relationship relates at least an effect of at least a nourishment identifier on the phenotype; and
determining the nourishment identifier as a function of the congenital relationship;
identifying, by the computing device, using the nourishment identifier, at least a nutrition element; and
generating, by the computing device, a consumption model using the at least a nutrition element.

12. The method of claim 11, wherein determining the nourishment identifier further comprises:
training a parameter machine-learning model with training data that includes a plurality of data entries correlating congenital factors to a plurality of congenital parameters; and
generating the congenital parameter as a function of the parameter machine-learning model and the at least a congenital factor; and
determining the nourishment identifier as a function of the congenital parameter.

13. The method of claim 11, wherein identifying the phenotype further comprises:
training a congenital classifier using training data which includes a plurality of data entries of congenital factors from a subset of categorized subjects;
classifying the congenital factor to the phenotype using the congenital classifier; and
identifying the phenotype as a function of the classifying.

14. The method of claim 13, wherein classifying further comprises classifying the congenital parameter to a nutrition-linked congenital disorder category.

15. The method of claim 11, wherein generating the congenital relationship further comprises:
generating a nutraceutical model using a machine-learning process and training data which includes a plurality of data entries correlating effects of nourishment identifiers to phenotypes; and
determining the congenital relationship as a function of the nutraceutical model and the phenotype.

16. The method of claim 14, wherein computing device is further configured to generate a nourishment training dataset from a plurality of congenital relationship outputs of the nutraceutical model.

17. The method of claim 11, wherein identifying the at least a nutrition element further comprises:
generating a nutrition model using training data including a plurality of data entries of nourishment identifiers correlating to nutrition elements; and
determining the at least a nutrition element as a function of the nutrition model and the nourishment identifier.

18. The method of claim 17, wherein generating the congenital nourishment program further comprises generating a linear programming function with the plurality of nutrition elements wherein the linear programming function outputs at least an ordering of the plurality of nutrition elements according to the consumption model.

19. The method of claim 11, where the consumption model comprises a nutrition-linked result.

20. The method of claim 11, wherein the congenital nourishment program includes a nourishment score.
